SEQUENCE LISTING (4 sequences)

SEQ ID NO: 1 - DNA, 20 bp, Artificial Sequence
  KIR2.1 1st sense
ccaaagcagaagcactggag

SEQ ID NO: 2 - DNA, 22 bp, Artificial Sequence
  KIR2.1 1st A/S
ctttgaaaccattgtgcttgcc

SEQ ID NO: 3 - DNA, 39 bp, Artificial Sequence
  KIR2.1 ICR HindIII sense
cactatagggaagctaccatgggcagtgtgcgaaccaac

SEQ ID NO: 4 - DNA, 36 bp, Artificial Sequence
  KIR2.1 ICR HindIII A/S
atagaataggaagcttcatatctccgactctcgccg

(19) United States
(12) Patent Application Publication
(10) Pub. No.: US 2021/0355418 A1
(43) Pub. Date: Nov. 18, 2021

(54) ELECTRODE HAVING NANO STRUCTURE AT TIP

(71) Applicants: Mitsuyoshi SAITO, Tokyo (JP); Ion Chat Research Corporate, Saitama (JP)

(72) Inventor: Mitsuyoshi Saito, Tokyo (JP)

(73) Assignee: Ion Chat Research Corporate, Saitama (JP)

(21) Appl. No.: 17/289,349

(22) PCT Filed: Oct. 29, 2019
(86) PCT No.: PCT/JP2019/042432

§ 371 Date: Apr. 28, 2021

(30) Foreign Application Priority Data
Oct. 29, 2018 (JP) 2018-203171

(51) Int. Cl. C12M 1/34; G01N 33/50

(52) U.S. Cl. CPC C12M 1/34; C12Q 1/02; G01N 33/5061; G01N 33/5088

(57) ABSTRACT

The purpose of the present invention is to provide a method which is designed to form an intracellular recording electrode for a cell by a simple operation which is less invasive to the cell and does not need a magnetic force, and with which the short-term or long-term intracellular potential can be accurately measured. More specifically, provided is a method comprising: securing, to a manipulator or the like, a holder provided on a conductor having a conductive nano structure at a tip; making the tip nano structure part penetrate a cell membrane while adjusting the amount of pressure applied to the target cell, thereby forming an intracellular recording electrode independently secured above the cell; and measuring the intracellular potential. The conductive nano structure at the tip and the conductor main body do not have to be magnetic but may be stuck together by magnetic force or may be formed as one body. When the cell membrane potential of a target cell cultured in a typical culture vessel is recorded, by forming the conductor main body of a magnetic electrode (MagEle) and independently securing same using a ring-shaped magnet that is provided on the lower surface of the culture vessel and that secures a light projection path or a light observation path through the center thereof, measurement of the intracellular potential of the target cell and fluorescent observation of changes in intracellular potential due to a light stimulus or intracellular calcium dynamics can be performed simultaneously.

### TECHNICAL FIELD

The present invention relates to an electrode having a nanostructure at the tip, a method of measuring an intracellular potential or a change in potential using the electrode, and a method of controlling the intracellular potential.

### BACKGROUND ART

All cells have different ionic compositions inside and outside the cell, and the intracellular potential (membrane potential) is maintained by a transporter (i.e., sodium pump) that keeps the difference in ion distribution and the difference in ionic composition. In the resting state, the membrane potential is stable (resting membrane potential); however, when the ion channels on the cell membrane surface are activated and the ion channels are opened, ions are released or flow in at once through the ion channels due to the difference in ion concentration inside and outside the cell membrane. This causes the electric potential of the entire cell changes where depolarization or hyperpolarization occurs, and consequently, the intracellular membrane potential changes. As a result, the generation/transmission of action potentials occurs in the myocardium and nerves in which then, an information transmission occurs, such as the release of hormones and neurotransmitters and the contraction of myocardial and skeletal muscle cells.

Conventionally, changes in cell membrane potential and accompanying measurement of membranes current through ion channels have been used to observe changes in the cell state and the cell response to drugs. In drug discovery screening, in particular, drug candidates are exposed to cultured cardiomyocytes, nerve cells and other cells, and changes in membrane potential are measured to assess cardiotoxicity and neurotoxicity.

In order to measure the intracellular potential, conventionally, a metal electrode or a micro glass electrode is filled with an electrolytic solution, inserted into the cell, and then the current or voltage is measured from the potential difference with the extracellular electrode. This measurement method called the patch clamp which has become the industry standard method. The patch clamp method precisely measures and controls intracellular potential changes by bringing a glass pipette filled with intracellular electrolyte into close contact with the cell membrane and electrically integrates the glass pipette and cells.

The patch clamp method is subdivided into two different recording modes. A whole cell mode for measuring the dynamics of ion channels expressed in the whole cell, and a method for measuring the dynamics of a single channel (single channel activity) that is contained in only the cell membrane within the inner diameter of the patch pipette (cell mode). There is also a method (inside patch, outside patch mode) in which the microcell membrane is separated from the cells and measured.

In the whole cell mode, changes in intracellular potential and dynamics of current (ion channel activity) passing through ion channels throughout the cell are measured by breaking through the cell membrane inside the electrode bonded to the glass pipette. The above procedure requires a highly skilled technique and a high degree of expertise because it is a recording method performed by using electrodes directly on individual cells under a microscope.

These intracellular recording methods (voltage-clamp, current-clamp) can observe the dynamics of how ions pass through ion channels in the cell membrane, as represented by the patch-clamp method (whole-cell patch-clamp). In the voltage-clamp mode, the feedback function included in the patch-clamp amplifier efficiently controls the intracellular potential, and the fast phenomenon that occurs in milliseconds due to the opening and closing of the ion channel can be recorded as an electric current change. In the current-clamp mode, the action on the cell due to the activity of the ion channel can be recorded as a (membrane) potential change.

The patch-clamp methods include manual patch-clamp method and auto patch method. Among those, the manual patch-clamp method has high reliability of data in electrophysiological measurement. However, the manual patch-clamp method is very inefficient because an operator uses a microscope to operate a manipulator to perform an experiment and requires a large amount of specialized knowledge. This has become a major hurdle in medical biology research, especially in the field of drug discovery.

On the other hand, the auto patch-clamp method uses an automated electrophysiological measuring instrument, and although its performance has improved remarkably in recent years, the reliability of data is not as reliable as replacing the manual patch-clamp method. Furthermore, the auto patch-clamp test equipment is so expensive that its use is limited to large pharmaceutical companies.

In recent years, a method similar to the patch clamp method was developed but high voltage is applied when penetrating an electrode into a cell membrane in order to measure an intracellular potential (Non-patent Document 3) which led to the development of rat myocardium. It has been reported that the intracellular potential of rat cardiomyocytes could be recorded successfully. However, in that method, the electrode loses access to the intracellular space, because the perforated cell membrane is repaired immediately and the maximum observable time of electrical response is about 10 minutes; therefore, it is not a practical method.

With the progress of intracellular recording methods, the development of extracellular recording methods for recording extracellular electrical changes has developed and become more widespread in recent years. The extracellular recording method, as represented by an in vitro multi-point planar electrode (multi-electrode array) system, records electrical changes extracellularly from electrodes placed outside the cell (Patent Documents 1 to 4).

The application of the in vitro multi-point planar electrode (Multi Electrode Array, MEA) system began to be used in studies of plasticity of cultured nerve cells, or more specifically, drug safety testing using nerve cells and cardiomyocytes derived from human iPS cells.

Although MEA allows for easier handling of cells due to extracellular recording, this technology only allows recordings for AC-like changes (changes in membrane potential unit time, differential waveforms), so it is not applicable for recording slow changes in the intracellular membrane potential. Therefore, this measurement method does not provide sufficient information for analysis and its application is limited.

According to a report that attempted intracellular recording by a method based on MEA, cells were seeded on a mushroom-shaped electrode and high voltage was applied in order to break the cell membrane. The recording time of the intracellular potential could only be maintained for a short time, within 3 minutes, confirming it is not a practical recording method. (Non-Patent Document 1).

Therefore, the present inventors have recently introduced conductive nanoparticles such as gold-coated magnetic nanoparticles instead of glass microelectrodes as a method for accurately and easily measuring and controlling the intracellular potential with less invasiveness to cells. We have developed a method for recording intracellular potentials and potential changes using extracellular electrodes. More specifically, by utilizing the action of a strong magnet to penetrate the intracellular (or cell surface) conductive nanoparticles through the cell membrane and connect the electrodes inside and outside the cell, the intracellular measuring device is used to connect the electrodes inside the cell. It has become possible to measure the potential and the change in the potential. This method has the surprising effect of observing the fluctuation of intracellular potential in living cells for a long period of time by suppressing damage to cells as much as possible, and as a result, it was internationally dated Apr. 27, 2018. A patent application was filed (PCT/JP2018/017334).

In addition, the present inventors have developed a “capacitive potential measurement device method” as a new intracellular potential measurement method. The capacitor referred to here is composed of a conductive plate for seeding cells, an insulator located below the conductive plate, and a second conductor measures the potential difference between the two conductors. According to this method, the conductive plate as a sensor senses the change in charge inside the cell through the conductive nanoparticles penetrating the cell membrane, performs charge-voltage conversion, and records it as the change in intracellular potential. Therefore, it is not necessary to set the ground for the extracellular solution, and the conductive plate may be in direct contact with the extracellular solution at a portion that is not in contact with the cells.

A patent application for this method was also filed on Apr. 27, 2018 (Japanese Patent Application No. 2018-87689).

### CITATION LIST

### Patent Document

Patent Document 1: WO2012/043820
Patent Document 2: WO2013/061849
Patent Document 3: WO2014/098182
Patent Document 4: Japanese Translation of PCT International Application Publication No. 2005-505761

### Non-Patent Document

Non-Patent Document 1: Anna Fendyur, et al., Frontiers in Neuroengineering, December 2011, Vol. 4, Article 14, p. 1-14
Non-Patent Document 2: Raphael Levy, et al., NanoReviews 2010, 1:4889-DOI: 10.3402/nano. v1i0.4889
Non-Patent Document 3: Micha E. Spira et al., Nature Nanotechnology Vol. 8(February 2013) p. 83-94/DOI:10.1038/NNANO.2012.265

### SUMMARY OF THE INVENTION

### Problems to be Solved

In the intracellular potential measurement method using conductive nanoparticles of the present inventors, by using conductive nanoparticles instead of conventional glass microelectrodes, there is little invasiveness to cells, and it is accurate and easy for a long period of time. It is a method that enables measuring intracellular potential over a long period of time; however, a magnetic field is required not only to allow the conductive nanoparticles to penetrate the cell membrane but also to fix the electrode to the cell surface, and there are some restrictions on the measuring device.

For example, when the intracellular recording electrode is formed below the cell, it is necessary to provide a magnet below the cell via a conductive plate in order to allow the conductive nanoparticles that are introduced into the cell to penetrate through the cell membrane. The inventor has found that by using a magnet electrode (Magele), it is possible to form an intracellular recording electrode by attracting intracellular conductive nanoparticles from above the cell, in addition, with this method, it is not necessary to introduce the intracellular conductive nanoparticles in advance. However, in the former case, in order to fix the magnet electrode (Magele) above the cell to the cell surface, and in the latter case, in order to attract the conductive nanoparticles on the surface of the magnet electrode and fix the magnet electrode, it was necessary to install an iron plate under the cell and utilize the magnetic field of the magnet electrode. That is, all of them were methods in which the use of magnetic force was indispensable in order to fix the electrode on the cell surface and form a stable intracellular recording electrode.

An object of the present invention is to provide a simple method for measuring intracellular potential, that consists of formation of an intracellular recording electrode without using magnetic force, that is an improved version of the intracellular potential measurement approached from above the cell, which offers lesser degree of cell damage, accurate long term intracellular potential measurement, and the simpler process of nanostructure cell membrane penetration.

### Means for Solving the Problems

The present invention is an improved version of the previous claimed invention by this inventor, the formation of a magnetic field is not required, that was essential in the previous invention for forming an intracellular electrode by penetrating conductive nanoparticles through the cell membrane of a target cell for recording the intracellular potential changes, and for self-sustaining fixation of the magnet electrode (Magele).

That is, it is not necessary for the method to install an iron plate or a magnet to generate a magnetic force below the cell, and a conductor having no magnetic force can be used instead of the magnet electrode (Magele) to be adhered from above the cell surface.

This specific method is to fix the intracellular recording electrode above the cell using a holder such as a manipulator whose degree of pressurization can be adjusted to its preferred pressure, and instead of the conductive nanoparticle-magnet electrode, a conductive nanostructure which is a metal that is 10-50 nm at the tip (i.e., an electrode made of a conductor having a Nanostructure or a nano-protrusion structure) is used.

The “electrode having a conductive nanostructure at the tip” of the present invention also includes the above-mentioned “magnet electrode (Magele) having conductive nanoparticles adsorbed at the tip”. In that case, in order to fix the conductive nanoparticles to the tip of the electrode, it is essential for the conductive nanoparticles to be attracted to the magnetic force. In order to place the electrode at an appropriate position on the cell surface, the magnetic force of the magnet electrode is not used, but instead, it is characterized by the electrode that is placed with a holder and a manipulator to fix the holder with uniform mechanical pressure.

To allow the conductive nanoparticles to penetrate the cell membrane when the conductive nanoparticles are attracted to the tip using a magnet electrode (Magele), it is necessary to form an intracellular potential recording electrode composed of “magnet electrode-conductive nanoparticles”. According to the method mentioned above, in the PCT application specification of the present inventors, two types of methods are disclosed to aspirating and penetrating conductive nanoparticles through a cell membrane:

(1) A magnet electrode in which conductive nanoparticles previously introduced into cells are brought into contact with the upper surface of cells. A method of attracting by (Magele) to penetrate the cell membrane (FIG. 1, left); and
(2) Conductive nanoparticles are adsorbed on the surface of the magnet electrode (Magele) in advance, and the conductive nanoparticles on the surface of the magnet electrode (Magele) are brought into contact with the upper surface of the cell and placed on an iron plate placed below the container to which the cell is adhered (FIG. 1, right).

In the present invention, a conductor having no magnetic force may be used instead of the magnet electrode (Magele), and instead of adsorbing the conductive nanoparticles on the magnet electrode (Magele), a nano-protrusion structure is formed at the tip thereof or bonded conductors can be used. According to the method of the present invention, even a non-magnetic conductor having various shapes of nano-protrusion structures are conductivity at the tip can penetrate the tip portion through the cell membrane. Moreover, it is a method that allows the above-mentioned electrodes to be placed in a fixed position while adjusting the pressure on the cell surface as a means for accurately and easily measuring the intracellular potential of a target cell by using an electrode having a conductive nano-protrusion structure at the tip obtained by the method as an intracellular recording electrode.

More specifically, this present invention uses a manipulator or the like that is installed above, the tip portion of a conductor having a conductive nanostructure at the tip is penetrated from the upper surface of the cell in a fixed state where the pressure on the cell is adjusted (FIG. 2). In the present invention, since the conductor body serves as an electrode, it can be moved up and down by a manipulator or the like to adjust the pressure on the cell; therefore, a magnetic force for fixing the electrode on the cell is not required. As a result, the conductor serving as the intracellular recording electrode of the present invention may be formed by using a conductive material having a nano-protrusion structure at the tip, and it is not necessary to use a magnet electrode (Magele). Furthermore, the nano-protrusion structure at the tip is preferably firmly fixed to the conductor of the electrode body, but it may be in a state of being adsorbed on the surface of the electrode that adheres to the cells. When a magnet electrode (Magele) is used as the conductor and a nanostructure of a conductive material is formed at the tip thereof (including the case where conductive nanoparticles are adsorbed), an iron plate placed under the cell together with a manipulator is used. The pressure load applied to the cells may be adjusted by using the magnetic force of.

In the present invention, other than the portion having the conductive nanostructure at the tip of the electrode, the outer surface is wrapped completely with an insulator and completely insulated from the external liquid, so that the grounding point can be installed arbitrary. By wrapping the outside of the insulator with a ground point, it can be used as a single electrode (FIG. 3).

Furthermore, by applying the “capacitive potential measuring device method” (Japanese Patent Application No. 2018-87689) developed by the present inventors, a compact “capacitive potential measuring device” with a holder (FIG. 3, right) can be manufactured. It can also be used as a “Clip on ground”.

The “Clip on ground” refers to recording using a magnet electrode (C-M electrode) to which a capacitive potential measurement device function is added. The magnet electrode (C-M electrode) covers the area other than the cell contact surface of the magnet electrode (Magele) with a parafilm, the positive electrode is directly connected to the magnet electrode, and the negative electrode is connected from above the parafilm. It refers to a device in which the magnet electrodes of the body are used as a capacitive potential measurement device. In the present invention, since it is not necessary to have magnetism, a conductor may be used as an electrode instead of the magnet electrode (Magele). Further, by providing a plurality of capacitive potential measuring device type electrodes on the conductive plate held by the holder, the plurality of electrodes can be operated at the same time (FIG. 4).

In addition, although it is described as a magnet electrode (Magele) and magnetic nanoparticles in FIG. 4, it may be an electrode in which a conductive nanostructure is formed at the tip of a non-magnetic conductor. It can also be used as a connecting electrode in which a magnet electrode (or a conductor) is connected in combination with a fixing magnet (FIG. 4).

When recording from cultured cells placed on a magnetic metal using a MagEle electrode, the magnets that make up the MagEle electrode have the effect of adsorbing conductive nanoparticles and pulling the magnetic iron plate below to make MagEle self-supporting (FIG. 4, FIG. 25).

By using a donut-shaped electrode having a ring-shaped magnet instead of the magnetic metal, MagEle can be fixed and self-supporting without a magnetic iron plate. In this method, by supporting MagEle with a ring-shaped magnet, it is possible to secure a path directly viewed from the center of the ring, and it is possible to directly project light onto the cultured cells existing below MagEle (FIG. 25 right, FIG. 26).

The donut-shaped electrode allows for, the following use to be considered: (1) It becomes possible to perform photostimulation on Channelrhodopsin-expressing cells (FIG. 26, left); and (2) By using the test cells into which the fluorescent reagent has been introduced, it is possible to simultaneously perform, such tasks like intracellular calcium dynamics, changes in membrane potential, etc. in parallel with intracellular potential measurement (FIG. 26, right).

That is, the present invention includes the following inventions.

[1] An intracellular recording electrode comprising a conductor having a nano-protrusion structure at the tip,
wherein the nano-protrusion structure penetrates the cell membrane of a target cell; and the conductor is installed at a fixed position on the upper surface of a cell and is subjected to mechanical uniform pressure from above.
[2] The intracellular recording electrode according to [1], wherein the conductor is a magnetic electrode (Magele) having conductive nanoparticles as the nano-protrusion at the tip or is a conductor containing a non-magnetic conductive substance and having a nano-protrusion formed at a tip.
[3] The intracellular recording electrode according to [1] or [2], wherein the conductor is integrated with a holder that holds the conductor.
[4] The intracellular recording electrode according to [3], wherein the holder is fixed to a manipulator capable of adjusting the degree of pressurization on the cell surface.
[5] The intracellular recording electrode according to any one of [1] to [4], wherein the entire side surface of the conductor body is covered with an insulator;

one or more positive poles are connected to the conductor body; and the region of the conductor in contact with the extracellular solution is covered with the insulator, wherein a negative pole is connected to a region that does not come into contact with the extracellular solution to form a potential recording circuit.

[6] An intracellular recording electrode comprising a magnetic electrode (MagEle), wherein the magnetic electrode has a nano-protrusion at a tip thereof, and the nano-protrusion portion at the tip penetrates a cell membrane of a target cell, wherein the magnetic electrode is set in position on a cell upper surface under mechanical uniform pressure from above or under magnetic force with a magnet or magnetic metal below a vessel; an entire side surface of the magnetic electrode is covered with an insulator; a plurality of positive poles are connected to the magnetic electrode; a region of a positive pole surface which makes contact with extracellular solution is further covered with an insulator material; and a negative pole is connected to an insulator surface in a region that does not make contact with extracellular solution; thus forming a multi-electrode capacitive potential measuring device with electrodes themselves constituting capacitors.
[7] An intracellular recording electrode forming a multi-electrode capacitive potential measuring device, wherein the multi-electrode capacitive potential measuring device has a capacitor formed between a positive electrode and a negative electrode; the positive electrode is connected to the surface of conductive glass electrode having the cultured target cells into which the conductive nanoparticles have been introduced in advance; and the negative electrode is connected to a conductive electrode composed of a plurality of metal foils that are placed on the lower surface of non-conductive side of conductive glass. Since only the upper surface of the conductive glass is subjected to the conductive treatment, the lower surface is not conductive.
[8] The intracellular recording electrode according to [7], wherein the electrode is characterized by electrode pairs formed by the positive electrodes and the plurality of negative electrodes, wherein the continuity of the conductive glass electrode conductivity connected to the positive electrode is cut off corresponding to each section position of the conductive electrode connected to the plurality of negative electrodes.
[9] The intracellular recording electrode, according to any one of [6] to [8], characterized in that the surface of the conductive glass is washed with an alkaline solution having a pH of 10 or more, followed by washing the conductive glass with ddH₂O at least twice to remove the alkaline solution.
[10] A method for measuring the intracellular potential of a target cell or its potential change, comprising the step of using the intracellular recording electrode of any one of [1] to [9].
[11] A method for treating a glass surface to promote cell adhesion to the glass surface, characterized by cleaning the glass surface with an alkaline solution having a pH 10 or more; and cleaning with ddH₂O at least twice to remove the alkaline solution.
[12] The method for treating a glass surface to promote cell adhesion to the glass surface according to [11], wherein the glass is FTO or ITO conductive glass. A conductive metal such as titanium can be used instead of the conductive glass.
[13] A method for measuring the intracellular potential and a change in the intracellular potential of a target cells using the intracellular recording electrode, wherein the nano-projection structure portion at the tip of a magnet electrode (Magele) penetrates the cell membrane above the target cells, wherein light stimulation is applied on the target cells from the light projection path in the center of a ring-shaped magnet installed on the lower surface of the cover glass to which the cells, that express the photostimulatory reactive substance, are adhered.
[14] A method for measuring the intracellular potential and a change in the intracellular potential of a target cells using the intracellular recording electrode, and measuring the intracellular calcium dynamics or the change in membrane potential of the target cells by observing the fluorescence emitted from the target cells through the fluorescence observation pathway in the center of a ring-shaped magnet installed on the lower surface of the cover glass adhered with the target cells, wherein the nano-projection structure portion at the tip of a magnet electrode (Magele) penetrates the cell membrane above the target cells, wherein the fluorescence emission is applied on the target cells from the fluorescence emission projection path in the center of a ring-shaped magnet installed on the lower surface of the cover glass to which the cells, that express the photostimulatory reactive substance, are adhered.

### Effect of the Invention

In the present invention, no magnetic force is required for the conductive nanoparticles to penetrate the cell membrane and for the electrodes to be self-fixed on the cell surface, and the intracellular potential and the potential change can be measured more easily.

Furthermore, in the present invention, compared to the previously developed method, damage to cells can be suppressed and fluctuations in intracellular potential can be observed while allowing cells to survive in a normal state for a long period of time. Furthermore, the design is compact including the measuring device, and it can be used as a “Clip on ground” by using a capacitive potential measuring device type electrode. In addition, by using a donut-shaped electrode with a ring-shaped magnet, MagEle can be fixed and self-supporting without a magnetic iron plate. Light can be directly projected onto the test cells using the central hole to stimulate light and observe light signals.

### BRIEF DESCRIPTION OF THE FIGURES

FIG. 1 illustrates conceptual diagram of a method by which gold-coated magnetic nanoparticles are attracted to the tip of a magnet electrode (Magele) by a magnetic force to form an electrode having a tip conductive nano-protrusion structure used for penetrating a cell membrane.

FIG. 2 illustrates conceptual diagram of a method of fixing an electrode having a conductive nano-protrusion structure at the tip of the present invention by penetrating a cell membrane using a manipulator.

FIG. 3 illustrates structure of an electrode having a conductive nano-protrusion structure at the tip of the present invention.

FIG. 4 illustrates an example using a potential measuring device using an electrode having a nano-protrusion structure at the tip of the present invention is shown. When the diameter of the magnet electrode is short, it is difficult to stand on its own by magnetic force alone, so a strong “reinforcing magnet” is placed to help the thin magnet electrode stand on its own. In that case, the reinforcing magnet also functions as a holder to the ground.

FIG. 5 Reference: FIG. 5 illustrates a conceptual diagram of an intracellular recording electrode using magnet electrode.

FIG. 6 Reference: FIG. 6 illustrates a result of intracellular action potential measurement in Nav1.5/Kir2.1-expressing HEK cultured cells, with neodymium magnet electrode (induction of action potential by membrane potential change).

FIG. 7 Reference: FIG. 7 illustrates a measurement of the endogenous outward current of CHO cells mediated by conductive nanoparticles penetrating the cell membrane of CHO cells cultured on a conductive plate electrode (by the voltage-clamp method).

FIG. 8 Reference: FIG. 8 illustrates a preliminary experiment of measuring the intracellular potentials based on the charge amplifier principle.

FIG. 9 Reference: FIG. 9 illustrates a conceptual diagram showing that the intracellular potential (voltage) changes can be measured as detecting the changes in the charges by using the principle of the charge amplifier.

FIG. 10 Reference: FIG. 10 illustrates recordings of spontaneous action potentials from iPS cell-derived cardiomyocyte cells using intracellular gold-coated magnetic nanoparticle electrodes introduced in advance based on the Principle of Charge Amplifier.

FIG. 11 Reference: FIG. 11 illustrates the recording of sodium ion channel opener induced intracellular action potentials from cardiomyocytes cultured on a conductive glass surface coated with a collagen lattice, which was measured by detecting the changes in intracellular ion concentrations as changes in electric charge and then converting it into a change in voltage for measurement.

FIG. 12A Reference: FIG. 12A illustrates a conceptual diagram of a method recording the intracellular potential using the principle of the charge amplifier by using the conductive nanoparticles adsorbed on the magnet electrode as the intracellular electrode to penetrate the cell membrane instead of using conductive nanoparticles introduced into the cell in advance. A cover glass is used for cell culture.

FIG. 12B Reference: FIG. 12B illustrates a conceptual diagram of a method recording the intracellular potential using the principle of the charge amplifier by using the conductive nanoparticles adsorbed on the magnet electrode as the intracellular electrode that penetrates the cell membrane instead of using conductive nanoparticles introduced into the cell in advance. A cell culture insert is used for cell culture.

FIG. 13A Reference: FIG. 13A illustrates the experimental result for NG108-15 cells in which neural differentiation was performed for 5 days. Spontaneous action potentials were recorded by placing the magnet electrode, pre-adsorbed with conductive nanoparticles (C-M electrode), on cells seeded on coverslips.

FIG. 13B Reference: FIG. 13B illustrates the experimental result for NG108-15 cells in which neural differentiation was performed for 2 days. Glutamic acid was administered to the extracellular fluid (final external fluid concentration 800 μM), and the response mediated by glutamate receptors was recorded. The baseline and glutamate response artifacts in the figure were removed.

FIG. 14 illustrates data of NG108-15 cells activated by 10 mM glutamate and measured by patch clamp.

FIG. 15 illustrates differentiated NG108-15 cells are activated by 10 mM Glutamate, and a magnet electrode (Magele) with gold magnetic nanoparticles adsorbed at the tip is inserted into a silicon tube to insulate it, and a ground function with aluminum foil is attached to the outside. An example being one in which the active potential was recorded by pressing the integrated electrode with a manipulator from above the cell.

FIG. 16 illustrates data obtained by activating differentiated NG108-15 cells with Veratridine and measuring with a magnet electrode (Magele) in contact with gold-coated magnetic nanoparticles penetrating the cell membrane on the cell surface, and an enlarged view of a part thereof.

FIG. 17 illustrates measured data in which differentiated NG108-15 cells were activated by a K+channel blocker, a magnet electrode (Magele) with gold magnetic nanoparticles adsorbed on the tip was pressed from above the cells with a manipulator, and action potentials were recorded. This is an enlarged view of a part of it.

FIG. 18 illustrates resting membrane potential was measured while pressing (pressurizing) a magnet electrode (Magele) to Titanium nanostructures which were adhered to the tip of undifferentiated NG108-15 cells from above the cells with a manipulator. The recorded membrane potential disappeared by releasing the pressurization (upper figure). When the same experiment was performed in the absence of cells, there was no change in the potential (see the figure below).

FIG. 19 illustrates measurement of action potentials of SH-SY5Y cells differentiated by retinoic acid (RA).

FIG. 20 illustrates multi-electrode structure using MagEle.

FIG. 21A illustrates a first multi-electrode structure using conductive glass electrodes.

FIG. 21B illustrates a second multi-electrode structure using conductive glass electrodes.

FIG. 22 illustrates a result of intracellular potential recording from NG108-15 neuronal cell line using 3-electrode MagEle.

FIG. 23 illustrates measurement of intracellular potential in iPS-derived cardiomyocytes by 2-electrode MagEle.

FIG. 24 illustrates measurement of intracellular potential in Nav1.5, Kir2.1-expressing HEK cell line using 3-electrode conductive glass electrode.

FIG. 25 illustrates light protrusion path secured by making the MagEle electrode self-supporting with a ring-shaped magnet.

FIG. 26 illustrates measurement of cell activation by light stimulation (left figure) and fluorescence signal observation with an inverted microscope (right figure), which can be recorded at the same time as intracellular potential measurement using a MagEle electrode self-supporting with a ring-shaped magnet.

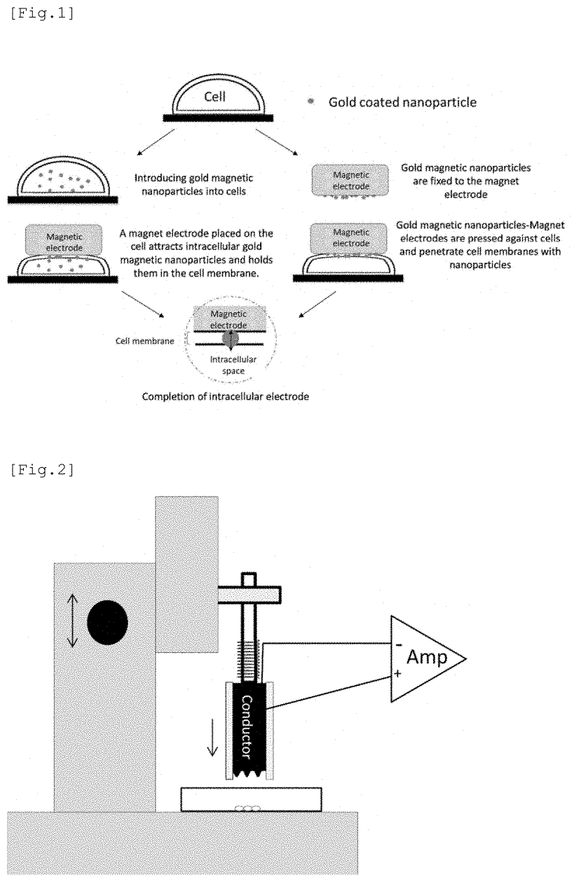

### DESCRIPTION OF EMBODIMENTS

### 1. “Electrode Having a Conductive Nanostructure at the Tip” of the Present Invention

### (1-1) Electrode Material

In the “electrode having a conductive nanostructure at the tip” of the present invention, it is sufficient that the conductor and the nano-protrusion at the tip form an electrode integrally, which can be achieved by either both being integrally molded, or being molded separately and then combined together. It is preferable that the conductive nano-protrusion structure is completely fixed to the tip of the conductor for easy handling, but it may be in a state of being adsorbed on the tip.

A magnet electrode (MagEle) may be used as the electrode material, but a conductor having no magnetism and no cytotoxicity (for example, various metals such as stainless steel, titanium, and gold, as well as conductive peptides and proteins, or various conductive polymers) may be used.

Here, the term “magnet electrode (MagEle)” refers to a magnet coated with a conductive material (for example, a conductive metal such as nickel or aluminum), or a typical one is a neodymium magnet, which has conductivity as well as magnetic force.

Neodymium magnets are the magnets with the highest magnetic force among permanent magnets, but they rust easily, so they are usually nickel-plated. Since a commercially available 1 mm diameter cylindrical neodymium magnet (Neomag Co., Ltd.) is also coated with Ni—Cu—Ni, it has high conductivity as well as strong magnetic force and can be used as a magnet electrode (MagEle). It can also be used as a magnet electrode (MagEle) even when it is coated with aluminum.

### (1-2) Electrode Structure:

The electrode for measuring intracellular potential used in the present invention has a “conductive nano-protrusion structure” at the tip portion in contact with the cell, and the “conductive nano-protrusion structure” penetrates the cell membrane to cause intracellular potential. It can act as an electrode that can record the potential.

Then, since the surface of the conductor such as the magnet electrode (MagEle) needs to be completely blocked from the extracellular fluid except for the contact portion with the cell, it comes into contact with at least the extracellular solution other than the contact portion with the cell. The area is preliminarily coated with an insulating coat such as silicon rubber or a silicon tube.

Furthermore, when providing a ground, wrap the surface of the insulating coat (parafilm, manicure, insulating paint, etc.) with a grounding material (silver wire, silver plate, aluminum foil, etc.), and use the conductor as a positive electrode on the surface of the grounding material and provide a negative pole.

In addition, a holder (holding body) is provided on the opposite side of the tip having the “conductive nano-protrusion structure” and is vertically attached to the manipulator if necessary. At this time, by providing a conductor having a plurality of conductive nano-protrusion structures on the conductive plate held by the holder and connecting each of them to the ground, the plurality of electrodes can be operated at the same time.

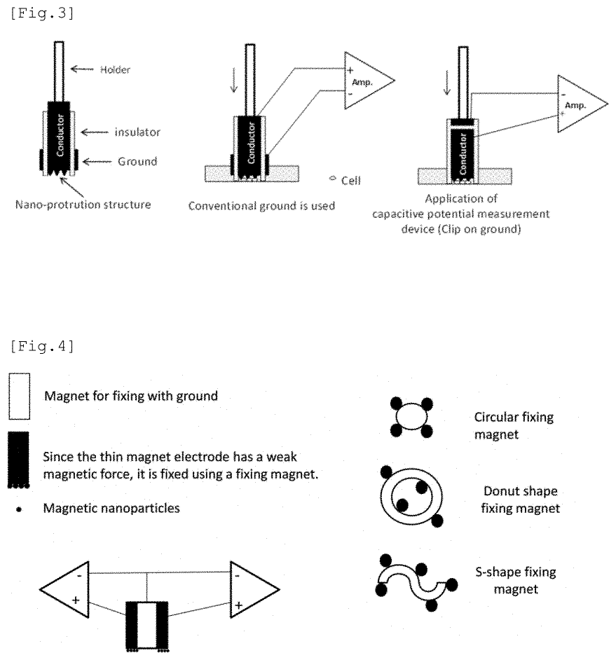

Recording can be performed by using the electrode of the present invention with “Clip on ground” which applies the principle of the capacitive potential measuring device. In this case, completely cover the surface other than the cell contact surface of the conductor such as the magnet electrode (C-M electrode), which is the electrode body, with an insulating coat such as parafilm, and connect the positive electrode directly to the conductor (magnet electrode). The negative pole is connected from above the parafilm (FIG. 3) and no grounding is required. In addition, each capacitive potential measuring device has a single holder or in via a conductive plate, and is attached to a manipulator as needed.

### (1-3) Application of Magele to Connect Composite (Multiple) Electrodes

In order to function as a connecting electrode, the electrodes of the present invention have magnet electrodes (Magele) attached to both sides of the fixing magnet, and the fixing magnet is surrounded by an insulating coat such as parafilm. It functions as a ground together with fixing the magnet electrode (Magele) (FIG. 4). When the fixed magnet is not used as a ground, it can also be used as a capacitive potential measurement device type.

In addition to this, as the connecting electrode, an electrode having a plurality of nano-protrusion structures can be used at the same time. The advantage of connecting electrodes is, for example, when (brain) nerve cells are cultured and protrusions extend from the cell body to form neural circuits, that by placing Mageles in multiple locations, it is possible to measure how neural circuits work with each other. In a normal Magele, the magnet electrode has the function of fixing gold magnetic nanoparticles on the electrode and the function of attracting the iron plate placed below by magnetic force to make the magnet electrode itself self-supporting. The size of the diameter of the magnet electrode (usually 6 mm) determines the number of cells to be recorded covered by the electrode. In order to reduce the number of cells to be recorded and improve the accuracy of the experiment, it is necessary to reduce the electrode diameter. At that time, since a cable connecting to the amplifier is attached to the electrode, if the diameter of the magnet electrode is reduced, the magnetic force decreases, and it becomes impossible to stand on its own by only the magnetic force with the iron plate. Therefore, a strong magnet (reinforcing magnet) is placed and attracted by magnetic force to help the thin magnet electrode become independent. At this time, at which the reinforcing magnet can also serve as a ground.

The reinforcing magnets can be of various shapes such as donut-shaped, plate-shaped, and S-shaped. Magnet electrodes or advanced nanostructure electrodes that can be attracted to the magnet can be placed at various positions of the reinforcing magnet, and potential recording can be performed from a plurality of positions, which is effective when analyzing a neural circuit. Currently, neural circuit in vitro analysis is limited to extracellular recording using multiple electrodes or using fluorescent reagents, so this is the first mechanism that enables simultaneous intracellular recording from multiple locations.

It should be noted that the plurality of electrodes can be fixed at a desired position at the upper part and act on the cells recorded by the manipulator without using a reinforcing magnet. It can also be applied to rat brain slice specimens (for example, in the hippocampus, simultaneous recording from CA1 and dentate gyrus).

Using a ring-shaped magnet as a reinforcing magnet, a rod-shaped magnet electrode (MagEle) installed directly above the open portion in the center of the ring is fixed and self-supporting without a magnetic iron plate. Light stimulation and optical signals can be observed by projecting light directly onto the test cells using the central open portion of the ring-shaped magnet.

### (1-4) Material and Shape of the Nano-Protrusion Structure at the Tip

The material of the tip portion does not need to have magnetism as long as it has conductivity. The tip portion may be molded integrally with the conductive electrode body, or it may be molded separately and bonded or adsorbed. When they are molded separately, they may be the same material or different materials. Any conductive material having conductivity and low cytotoxicity may be used, but preferable conductive materials include various metals such as stainless steel and titanium, conductive peptides and proteins, and various conductive polymers.

Furthermore, it is not necessary for the conductive material to be uniformly molded, and even if the core material is not conductive, particles whose surface is coated with the conductive material can be used. Examples of suitable materials for coating include, but are not limited to, conductive metals, such as gold and platinum (Yamada et al. (2015) WIREs Nanomed Nanobiotechnol 2015, 7: 428-445. Doi: 10.1002/wnan.1322), conductive peptides and proteins, or various conductive polymers. The “conductive nano-protrusion structure” of the present invention is a non-magnetic structure. Since various types of gold-coated magnetic nanoparticles, approximately 50 nm citric acid or PEG gold coated magnetic nanoparticles (manufactured by nanoimunotech, NITmagold Cit or PEG50 nm), etc., are commercially available and easily available, these gold-coated magnetic nanoparticles can be utilized for the formation of conductive nanoparticles. These magnetic nanoparticles can be used as the “conductive nano-protrusion structure” of the present invention. The conductive polymer and the conductive peptides: “Poly (anthranilic acid) with magnetite nanoparticles achieves enhanced crystallinity, magnetic properties, and AC and DC conductivity” (Ramesan and Jayakrishnan, 2017, SPE Plastic Research On line 10.2417/spepro.006898). In addition, Quantum dot (Qdot) particles (OO Otelaja, D.-H. Ha, T. Ly, H. Zhang, and RD Robinson, “Highly Conductive Cu2-xS Nanoparticle Films, which have been conventionally used for staining of biological imaging through Room Temperature Processing and an Order of Magnitude Enhancement of Conductivity via Electrophoretic Deposition” ACS Applied Materials and Interfaces 6, 18911-18920 (2014)) and the like which have been conventionally used for staining biological imaging can also be used as the conductive nanoparticles.

The shape of the nano-protrusion structure may be an appropriate shape, such as a sphere, an ellipse, a needle, or a rod. These are sometimes referred to as “conductive nano-protrusion structures”. The maximum length of the nano-sized protrusion structure needs to be longer than the thickness of the cell membrane (about 20 nm) because it needs to penetrate the cell membrane, but it should not be too long in order to minimize damage to the cells.

That is, in general, the maximum length (diameter in the case of a sphere) of 25 to 100 nm, is preferable. If possible, narrowing the numerical range to 30 to 80 nm, more preferably 35 to 70 nm, and even ideally 40 to 60 nm would provide the best results.

The electrode having a nano-protrusion structure at the tip of the present invention can be used as a terminal (“in vivo prep”) for direct contact with tissues, organs, etc. of a living body in clinical examinations for ALS and muscular atrophy. In that case, since it is necessary to make the advanced nano-protrusion structure reach the target living tissue, organ, etc., the maximum length is preferably 100 nm or more. If the measurement is performed in a state where the target tissue or organ is exposed during surgery, such as excision of a pathological site, it is used within the above general numerical range.

### (1-5) Method of Forming a “Conductive Nano-Protrusion Structure” at the Tip of an Electrode:

Hereinafter, typical methods for forming a “conductive nano-protrusion structure” at the tip of the electrode will be described, but the method is not limited to these procedures.

For example, similar to the method of pressing against the cell surface using the previous magnet electrode (Magele), a method of adsorbing a “conductive nano-protrusion structure” on the tip of a conductor and applying pressure while adjusting the degree of pressurization with a manipulator or the like is also included.

### (A) Method of Fixing Gold-Coated Nanoparticles to the Tip of an Electrode

Since the presence or absence of magnetism does not affect the present invention, a case where commercially available citric acid-stabilized magnetic gold nanoparticles (manufactured by nanoimunotech) will be described as an example.

Gold nanoparticles and L (+)-ascorbic acid are mixed at a ratio of 1:1 and dropped onto the tip of the conductor, and vapor deposition is performed while heating. When forming a nano-protrusion structure on a plate-shaped electrode, the electrode is laid down, and when it is a rod-shaped electrode, the electrode is inserted into a tube, and the above mixed solution is dropped into the space formed by the tube on the electrode to control the temperature. Hold on a hot plate adjusted to 75° C. for 15 minutes for vapor deposition, and then, it is washed with pure water.

### (B) Method of Fixing Nano-Sized Conductive Polymers or Peptides to the Tips of Conductive Electrodes

A case where Polyaniline Nanofibers is used as the conductive polymer will be described as an example. By using Chiou et al.'S “the dilute in situ deposition method” (Nature Nanotechnology 2, no. 6 (June 2007): 354-57.) With some modifications, Polyaniline Nanofibres can be used as the “conductive nano-protrusion structure” at the tip of the electrode to form nanostructures from 50 nm to 65 nm.

More specifically, the ratio of aniline to APS (ammonium peroxydisulfate) is 1.5 times. Polyaniline Nanofibers are formed on a titanium plate by mixing and reacting to a ratio ([aniline]/[APS]=1.5). A titanium plate having the obtained Polyaniline Nanofibers on its surface is used as it is as an electrode or bonded to the tip of the electrode instead of the titanium plate, conductive glass, a gold-coated glass plate, or the like may be used.

### (C) Method of Forming a Nano-Protrusion Structure of the Same Material on the Tip Portion of a Conductor:

Hereinafter, a method of forming a titanium crystal at the tip when the conductor is titanium will be described.

The method of Suzuki et al. (Journal of the Ceramic Society of Japan 117 (3): 381-384 (2009)) was partially modified and used.

In Suzuki's method, the titanium (IV) bis (ammonium lactate) dihydroxide solution of solution A and the TiO₂ colloidal solution of solution B are alternately sprayed on the titanium surface treated with KOH, including washing with water. This is a splay-LbL method in which titanium crystals are grown and uniform coating (TiO₂ anatase thin films) is performed in about 20 cycles.

On the other hand, since the object of the present invention is to form nanostructures of crystals having irregularities, the spray cycle is stopped from 5 to 10 times to cause irregularities of nanostructures on the coated surface. These irregularities perform the same function as gold magnetic nanoparticles (GMNP). The advantage of this method is that the number of cycles affects the shape and size of the crystal, so the nanostructure can be changed depending on the shape of the cell or tissue that records the potential.

### (1-6) Coating Material for Promoting Cell Membrane Penetration

Polyethyleneimine (PEI), which is a typical transfection reagent, can promote the process of penetrating the cell membrane even in the conductive nano-protrusion structure of the present invention. The conductive nano-protrusion structure at the tip of the electrode may be coated with PEI in advance, or PEI may be present on the cell surface when penetrating the cell membrane.

Any substance that has an effect as a transfection reagent for cells can be used in the same manner. For example, Superfect (Qiagen), plant lipids (azolectin) with electrical properties (20% negative charge) similar to cell membranes, or lipids such as 1,2-dioleoylphosphatidyl-glycerol (negatively charged DOPG Avanti) and 1,2-dioleoyl-sn-glycero-3-phosphocholine (charge neurtal DOPC, Avanti) mixed in a ratio of 1:4 have a similar effect.

### 2. Method of Penetrating the Tip of the Electrode of the Present Invention and Fixing it to the Cell Surface:

It is necessary to penetrate the nano-protrusion structure portion of the electrode tip of the present invention through the cell membrane and fix it to the cell surface as it is. At that time, since it is not necessary to fix the electrodes by magnetic force, neither an iron plate nor a magnetic plate is required below the cells.

In the present invention, the conductive nano-protrusion structure portion at the tip of the electrode is pressed from the upper surface of the cell to penetrate the cell membrane and fixed in a state where the pressure on the cell is adjusted. On the opposite side of the cell contact surface, it is preferable to provide a fixing device, such as a holder for, facilitating the adjustment of the pressure on the cells (FIG. 3).

At that time, since it is necessary to pressurize the cell membrane so as not to penetrate the cell and crush the cell itself, great care must be taken in performing the procedure. The pressure applied at that time is 1 kg or less, and intracellular recording can be obtained at a pressure of about 250 g or more. The stronger the pressure, the stronger the adhesion between the electrode surface and the cells, which is advantageous for recording the intracellular potential, but in the case of iPS-derived myocardium, there is a risk that the cells will be crushed and the nano-protrusions will penetrate the cells themselves. In order to avoid such danger, it is preferable to install a manipulator or the like above the electrodes and operate the manipulator attached to the electrodes via a spring structure while adjusting the degree of pressurization.

In addition, when a conductive nano-protrusion structure having magnetism, such as magnetic nanoparticles, is bonded or attracted to the tip of a magnetic material, such as a magnet electrode (Magele), it is placed between a metal plate, such as an iron plate placed below the cell. It can also be fixed using magnetic force.

More specifically, a magnet-attractive metal plate, such as an iron plate, is provided under the cell culture vessel, and the magnet electrode brought into contact with the upper part of the cell is attracted to the iron plate below the culture vessel to be fixed on the cell at the same time. The nano-protrusion structure at the tip of the electrode penetrates the cell membrane and enables intracellular recording.

Furthermore, the nano-protrusion structure at the tip of the electrode is coated with a substance for promoting cell membrane penetration, such as PEI described in (1-5) above. By allowing PEI, or the like, to be present on the cell surface in contact with the tip, the cell membrane penetration process is promoted.

### 3. Target for Measuring Intracellular Potential Using the Electrode of the Present Invention

In the method using the electrode of the present invention, the measurement can be performed by pressing the conductive nano-protrusion structure portion at the tip from the upper surface of the cell. Therefore, the measurement target is wide, and besides colonies of single cells and their aggregates, it is possible to directly measure the intracellular potential or potential change by directly contacting a tissue derived from a living body or a part of the skin, muscle, or other organ of the living body.

Hereinafter, the case of cells (including a cell population) separated from the living body such as cultured cells and living body-derived cells, and the tissues constituting the living body, cells in organs, and tissues will be described.

(3-1) Cultured Cells and Cells Derived from Living Body

The cells to be measured in the present invention may be cells of biological origin such as cells collected from biopsies, or cultured cells. It is mainly intended for mammalian cells, such as humans, but may be eukaryotic microorganisms, such as yeast, prokaryotic microorganisms, such as Escherichia coli, as well as birds, fish, and insect cells.

In particular, Cardiomyocytes, nerve cells, vascular epithelial cells, liver cells and the like, or cell populations thereof derived from human stem cells, such as human iPS cells are preferred.

In addition, transformed cultured cells, that express various ion channel genes or transporter genes using mammalian cells such as HEK and CHO cells as transformation hosts, are preferred target cells in the present invention since it can be used as an evaluation system for a toxicity test of a drug incorporated from various ion channels or transporters.

The cell to be measured in the present invention may be a single cell, or may be a cell proliferated after cell culture or a cell population (cell group) formed during the culture.

Since the present invention described here does not require magnetic force, it is not necessary to cover the entire bottom surface of the measuring container with cells. It is also possible to measure the intracellular potential in a single cell by blocking the region other than the contact surface of the electrode with the target cell from the extracellular fluid with an insulator coated on the electrode surface. On the other hand, when observing a cell population, it is necessary to seed the cells at a position where they are blocked from extracellular fluid by an insulator.

In the present invention, the term “cell population” refers to sheet-like cells formed on the surface of a culture dish (plate, well) for adherent culture, including cell clusters formed by cardiomyocytes, nerve cells, etc. derived from stem cells such as the iPS cells.

Additionally, a target cell of the present invention includes an artificial cell containing a giant liposome which has been widely used as a model cell in recent years (Moscho et al. (1996) PNAS 93: 11443-11447; Schlesinger Saito (2006), Cell Death. and Differentiation 13, 1403-1408; Aimon et al. (2011) PLoS ONE 6(10): e25529. doi: 10.1371/journal.pone.0025529).

For example, a method of fusing cell membrane fragments containing ion channels separated from cells expressing ion channels with giant liposomes, or artificial cells prepared by a method of fusing small liposomes incorporating a recombinant ion channel protein expressed in Escherichia coli or the like with giant liposomes can be used.

### (3-2) Model Cells Useful for Drug Discovery Screening

The present invention is particularly useful for drug discovery screening using model cardiomyocytes or nerve cells.

In drug discovery screening, by using the model cardiomyocytes and model nerve cells mentioned above, it is possible to analyze the effects of the test substance quickly and accurately on cell function, contractile activity due to electrical stimulation, and changes in electrophysiological characteristics. It is effective in evaluating the test substance by promptly evaluating the cytotoxicity and drug efficacy of the test substance.

The following is the preferred cardiomyocyte model. Cardiomyocytes that have been induced to differentiate from stem cells, such as human iPS cells, or cells in which the cardiac muscle ion channel genes including SCN5A (Nav1.5), CACNα1C (Cav1.2), KCNH2 (hERG), KCNQ1/KCNE1 (LQT1) and KCNJ2 (Kir2.1) have been introduced into cultured animal cells (HEK293, BHK, or CHO cells) and the above ion channels have been expressed in the cell membrane. As such cells, for example, myocardial model cells described in WO2014/192312 can be used.

Furthermore, instead of the transformed cells, a cultured cardiomyocyte sample in which differentiation is induced from stem cells, such as iPS cells, can be used (WO2014/192312). Myocardial iPS cells (iCell Cardiomyocytes) are also commercially available from CDI.

It is also possible to use a tissue section sample derived from a living body. At that time, as a tissue section, a myocardial section that forms atria or ventricles derived from mammalian cells was used to investigate the cause of atrial fibrillation and arrhythmia. A tissue piece, or the like, obtained by a biopsy from the diseased tissue can be used.

For a nerve cell model, a photoreceptor channel expressing cell obtained by introducing a photoreceptor channel gene into a nerve cell differentiated from PC12 cells or cerebral cortex cells, or iPS cells, can be used by observing the potential response to photo-stimulation. After the addition of the test substance, it becomes possible to evaluate the cytotoxicity of the test substance to nerve cells and to evaluate the drug efficacy. The light-sensitive model nerve cells, channel opsin 2-expressing cerebral cortical nerve cells described in JP-A-2006-217866 can also be used.

(3-3) Cells in Tissues and Organs that Make Up a Living Body

The electrodes of the present invention can act as terminals (“in vivo prep”) that come into direct contact with tissues, organs, etc. of a living body. In particular, it is useful for measuring cells and tissues that are relatively close to the surface of a living body, such as skin epithelial cells and muscle tissues. It can be used mainly as an alternative test method for needle electromyography.

It can also be applied to cells of tissues derived from biopsy specimens.

For example, in clinical trials, application in the following cases is preferable.

1. For neuromuscular junction diseases such as myasthenia gravis, continuous stimulation test and single muscle fiber EMG,

2. In neurogenic muscular atrophy, reflecting the loss of motor units during voluntary contraction and the subsequent compensatory expansion of remaining motor units, confirmation of increase in the potential itself and extension of duration with decrease in discharge frequency of motor unit action potential,

3. In motor neuron disease that damages anterior horn cells, the presence or absence of spontaneous discharge at rest,

4. In myogenic muscular atrophy, that is, myopathy, if a decrease in motor unit action potential and a decrease in duration are observed, atrophy and degeneration of the muscle fiber itself can be suspected.

The present invention is used to investigate the cause of such diseases. Accuracy applied to basic clinical trials to diagnose how diseases affect the expression and function of ion channels that make up the action potential by recording the action potential of the patient's muscle as an intracellular potential change (Reference: Muscle disease (Tokyo Metropolitan Neurological Hospital Shiro Matsubara http://www.jsnp.jp/pdf/cerebral_19.pdf).

As described above, since the muscle tissue is close to the skin surface, the muscle tissue can be directly inspected as (“in vivo prep”) by using the electrode of the present invention as a terminal, so that it can be applied to clinical examination of ALS and amyotrophic lateral sclerosis.

By directly measuring the potential change on the surface of the heart during surgery, it is possible to identify the area of the tissue necrotic and the area of dysfunction caused by myocardial infarction. Additionally, it may be possible to inspect on-site how the progression of the disease affects the shape and transmission of action potentials.

4. Method for Measuring Intracellular Potential Using the Electrode of the Present Invention and a Measuring Device for that Purpose.

In the measurement method using the electrode of the present invention, after the conductive nano-protrusion structure portion at the tip is pressed from the upper surface of the cell to penetrate the cell membrane, it is a general measurement conventionally used for intracellular potential measurement that can be connected to a device and measure intracellular potential and potential changes.

### (4-1) Container for Measuring Intracellular Potential

In the present invention, when the cultured cell is used as the intracellular potential measurement target, no magnetic force is required, so the bottom surface of the measurement container does not need to be a conductive plate. The culture vessel used for culturing can be used as is, but it is preferable to transplant from the container used for culturing to another container for measuring intracellular potential because it is easier to measure. At that time, the culture solution is washed with physiological saline multiple times to replace it with physiological saline.

### (4-2) Instrument for Measuring Intracellular Potential: In Normal Mode −1

In the present invention, the conductive plate electrode or magnet electrode that reflects the intracellular potential or current or the induced potential or current through the conductive nanoparticles penetrating the cell membrane is used. The current flow between the earth electrode provided in the extracellular fluid is sensed and recorded extracellularly. Since the current and potential generated at that time are extremely weak, a device equipped with an amplifier (amplification amplifier) for current, voltage, etc. is indispensable for recording the intracellular potential outside the cell.

Any device, any device conventionally used for measuring the intracellular potential or extracellular potential of a cell or a cell group can be used.

More specifically, if it is a patch-clamp amplifier or an intracellular recording amplifier, an amplifier having an input resistance of at least 10⁶ to 10⁸ ohms can be used. For example, a patch-clamp amplifier (Axopatch 200A, Axon instruments) or the like can be used. Moreover, if it is an MEA system, any device that can measure a DC signal instead of an AC signal can be used.

### (4-3) Method for Measuring Membrane Current and Equipment for it: In Normal Mode −2

A patch-clamp amplifier is used to measure the membrane current (current flowing through the ion channel existing in the cell membrane). In experiments using multiple artificial lipid bilayer membranes, the total membrane volume increases, so unlike the case of (4-2), the Axopatch-1D Patch-Clamp Amplifier (Axon Instruments) is used. Then, the Voltage clamp mode is used instead of the current clamp mode when the cell membrane potential is recorded.

The general patch-clamp amplifier Axopatch 200A (Voltage clamp) is particularly suitable for experiments in which the membrane potential of a normal single cell is controlled. When used for a large number of cells, the total area of the cell membrane-lipid bilayer is large, so it is necessary to control a large amount of membrane potential. Axopatch-1D, which can be used for the CV-4 Head stage for experimental measurement of artificial lipid bilayer membranes, was used. Since the Head stage for experimental measurement of artificial lipid bilayers can carry a large current, it has the advantage of being able to quickly charge the lipid bilayers of a large number of cells. A Bilayer Clamp Amplifier (BC-535) (Warner Instruments) or the like may be used instead of the Axopatch-1D patch-clamp amplifier.

### (4-4) Use of “Capacitive Potential Measuring Device”: In High-Sensitivity Mode

When a “capacitive potential measuring device” (capacitor formation) is used as the electrode of the present invention, more specifically, the electrode other than the cell contact surface of the electrode having a conductive nano-protrusion structure at the tip is completely covered with parafilm. The positive electrode is directly connected to the electrode, and the negative electrode is connected from above the parafilm. Since this negative pole acts as a normal ground, it is also referred to as “Clip on ground” in the present invention (FIG. 3).

In the present invention, the “capacitive potential measuring device” regards a change in intracellular potential such as an action potential occurring in a cell as a change in total ion charge. By applying the principle of the charge amplifier, this change in electric charge is converted into a voltage signal (intracellular membrane potential) and measured, and it is not necessary to provide a ground in the measuring circuit. (At that time, use a capacitor with very high impedance and picofarad capacitance. The potential change (in and out of charged ions into and out of the cell) that occurs at one end of the capacitor coupled to the conductive nanoparticle electrode is detected as a voltage because the high impedance capacitor acts as a charge-voltage converter (charge amplifier)).

The feature of this device is that the positive and negative poles of the amplifier are connected to both ends of the capacitor to directly measure the potential change. Since the negative pole in the device acts as the ground of the measurement circuit, it does not become an open circuit and enables stable measurement.

In addition, when referring to “a method of utilizing the principle of a charge amplifier” in the present invention, “Changes in intracellular ion concentration due to inflow and outflow of cations and anions through the cell membrane are used as changes in charge through a conductor such as a conductive plate in contact with the conductive nano-protrusion structure at the tip of the electrode that penetrates the cell membrane. It means a method based on the principle of sensing and measuring the change in electric charge as a change in voltage”.

As the conductive plate, conductive glass or titanium plate can be used, and as the negative electrode, aluminum foil, silver plate, platinum plate or the like is used.

The measuring method of the present invention is significantly different from the existing intracellular or extracellular potential measuring method. Instead of directly measuring the potential change that occurs between the positive pole (intracellular) of the amplifier and the negative pole placed in the extracellular fluid, charge-voltage conversion is performed using a capacitor (conductive plate) that seeds the cell as a sensor. It is not necessary to set the ground for the extracellular fluid because it is performed and recorded as the intracellular potential change. It suffices if the conductive plate serving as a sensor can sense the change in charge inside the cell through the conductive nanoparticles that have penetrated the cell membrane. Therefore, the conductive plate may be in direct contact with the extracellular solution at a portion not covered by cells. This method is particularly suitable for measuring the intracellular potential of a single cell such as cultured nerve cells that cannot be formed into a sheet.

In order to perform electrical stimulation to control the intracellular potential in the target cell, it is necessary to provide an electrical stimulation circuit with a ground separately from the capacitive potential measurement device, or to express Channelrhodopsine and perform photostimulation.

A typical electrode of the capacitive potential measuring device type of the present invention, as shown in (FIG. 2, right), has a structure in which the outside except for the tip portion in contact with cells is completely wrapped with an insulator and has a supporting portion (holder), and has a positive electrode, is directly connected to the electrode body, and the negative electrode is connected from above the parafilm.

### (4-5) Method for Measuring Intracellular Potential of Cells in a Tissue or Organ of a Living Body by “In Vivo Prep”

The “in vivo prep” for measuring the intracellular potential of cells in living tissues or organs is an application of an intracellular potential recording device via conductive nanoparticles. More specifically, among the conductors of the present invention shown on the right side of FIG. 3, the outside of the conductor excluding the terminal at the tip that comes into contact with the cell is completely wrapped with an insulator such as parafilm, and the outside is covered with a ground electrode (mode 1). In addition, the intracellular potential can be recorded as a capacitive potential measuring device using a capacitor formed by a second conductor sandwiching the electrode recording portion and the insulator (mode 2).

In both mode 1 and mode 2, as shown in FIG. 4, a plurality of electrodes can be connected to a plurality of amplifiers as potential measuring devices, and a plurality of intracellular potentials can be recorded at the same time.

In general, “in vivo prep” is directly contacted with cells in tissues, organs, or cells from a biopsy sample to measure the intracellular potential. When recording from tissues that are relatively close to the surface of the living body, such as skin epithelial cells and muscle tissue, by setting the length of the conductive nano-protrusion at the tip to 100 nm or more, it is possible to measure the intracellular potential through epithelial cells, fascia, and the like.

In addition, if the heart is opened during surgery, the intracellular potential change on the surface of the heart can be directly measured.

(4-6) How to Use a MagEle Electrode that is Self-Fixed by a Ring-Shaped Magnet

The role of the magnet when measuring the intracellular potential using the MagEle electrode is to attract conductive nanoparticles that have penetrated the cell membrane of the cultured cell and to adsorb the magnetic metal (magnetic iron plate) placed under the cultured cells and make MagEle self-supporting. Like magnetic metals, ring magnets can also self-fix MagEle.

At that time, it is possible to secure a path for directly looking at the cultured cells to be examined below the MagEle from below the center of the ring-shaped magnet, and it is possible to directly project light onto the cells.

Taking advantage of this, for example, Channel rhodopsin can be expressed in test cells in advance, and the cells can be light-stimulated to record the intracellular potential change. By introducing a fluorescent reagent into the test cells, it becomes possible to perform intracellular calcium dynamics, changes in membrane potential, etc. in parallel with electrical measurement using the MagEle electrode.

If the culture vessel can be installed on a table onto which a light irradiation path can be secured, the vessel Light stimulation or fluorescence observation by fixing the electrode with a manipulator, or the like, using the HandyNano technology without installing a ring-like magnet below, and by fixing the electrode with a manipulator, or the like, in combination with the HandyNano.

### EXAMPLES

Examples will be shown below, and the present invention will be more specifically described; however, the present invention is not limited thereto.

Other terms and concepts in the present invention are based on the meanings of terms commonly used in the art, and various techniques used to carry out the present invention particularly including techniques whose sources are clearly stated. Except for this, those skilled in the art can easily and surely carry out the procedure based on known documents and the like.

In addition, various analyses were performed according to the methods described in the analytical instruments or reagents used, the instruction manuals for the kits, catalogs, and the like. The technical documents, patent gazettes, and patent application specifications cited in the present specification shall be referred to in the description contents of the present invention.

### (Reference Example 1) Measurement of Intracellular Potential in HEK Cells that Stably Express Nav1.5/Kir2.1

In this experiment, it is shown that the intracellular potential can be measured by conductive nanoparticles penetrating the cell membrane using HEK cells that stably express Nav1.5/Kir2.1 that spontaneously generate action potentials.

### (Reference 1-1) Preparation of Stable Expression HEK Cells of Nav1.5/Kir2.1

HEK cells (JCRB cell bank) were transformed with the Nav1.5/Kir2.1 gene vector to prepare HEK cells that stably express Nav1.5/Kir2.1. Specifically, the Nav1.5 gene was first excised from the a subunit of human Na+channel (Nav1.5) (SCNA5, BC1 40813: SourceBioscience) and inserted into pcDNA3.1 (−) hygromycin (Invitrogen).

Since BC140813 is an Embryonic type Nav1.5 gene, it was replaced with the Nav1.5 gene expressed in human adult myocardium by PCR using human heart cDNA (Zymogen). The Kir2.1 (NM_000891, KCNJ2) gene (1284 bp) was cloned using the Nesting PCR method using the following primers after synthesizing cDNA from total RNA extracted from iPS cell-derived cardiomyocytes (CDI, Cellular Dynamics International) using reverse transcriptase.

Kir2.1 1st sense: CCAAAGCAGAAGCACTGGAG (SEQ ID NO: 1)

Kir2.1 1st A/S: CTTTGAAACCATTGTGCTTGCC (SEQ ID NO: 2)

Since the PCR product could not be detected by the first round PCR using the above primer, the PCR reaction was diluted 100-fold and further PCR (Nesting PCR) was performed to obtain the Kir2.1 gene.

Kir2.1 ICR HindIII sense: CACTATAGGGAAGCTACC atgggcagtgtgcgaaccaac (SEQ ID NO: 3)

Kir2.1 ICR HindIII A/S: ATAGAATAGGAAGCT tcatatctccgactctcgccg (SEQ ID NO: 4)

The obtained Kir2.1 PCR product was inserted into the HindIII site of pD608 (blastcidin, DNA2.0). The Kir2.1 gene (2ug/ml blastcidin) was introduced into HEK293 cells (culture medium, DMEM, Sigma-Aldrich, 10% FBS) together with the Nav1.5 gene (50ug/ml hygromycin). A stable expression cell line of Nav1.5/Kir2.1 was established.

### (Reference 1-2) Introduction of Gold-Coated Magnetic Nanoparticles into Cells

Gold-coated magnetic nanoparticles were introduced into “HEK cells stably expressing Nav1.5/Kir2.1” prepared in (Reference 1-1) using Polyethyleneimine (PEI, P3143 Sigma-Aldrich). The protocol used was improved based on the protocol reported at the URL below (https://labs.fccc.edu/yen/docs/PEI %20preparation.pdf).

Specifically, a 10 mg/ml PEI solution (pH 7) is filtered by 0.2 μm (Minisa rt, Sartorius stedim) in advance, and then stored at −80° C. Immediately before use, the amount used was diluted 100-fold with ddH₂O. A 5 μl PEI diluted solution was added to 80 μl gold-coated magnetic nanoparticles and 20 μl 5×HBPS (24 mM KCl, 1 mM CaCl₂), 1 mM MgCl₂, 10 mM Glucose) mixture and incubated for 15 minutes at room temperature.

Then, the HEK cells stably expressing Nav1.5/Kir2.1 prepared in (Reference 1-1) were washed with DMEM (manufactured by Sigma-Aldrich) containing no serum, or OptiMEM I (manufactured by Invitrogen)-containing buffer solution (PBS). The HEK cells were then incubated in the gold-coated magnetic nanoparticles solution above for 15 minutes in an incubator at 37° C.

The obtained gold-coated magnetic nanoparticles-introduced “stable expression HEK cells of Nav1.5/Kir2.1” were cultured in a normal culture dish (DMEM, Sigma-Aldrich, 10% FBS).

### (Reference 1-3) Method of Attracting Intracellular Conductive Nanoparticles with an Upper Magnet Electrode (MagEle) and Penetrating the Cell Membrane

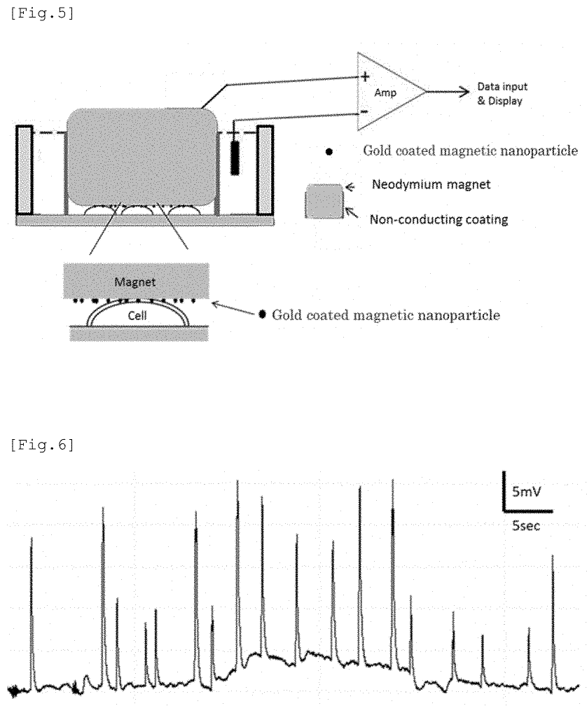

This experiment shows that it is possible to construct an intracellular recording electrode using a 1 mm diameter cylindrical neodymium magnet (Neodymium Co., Ltd.) coated with Ni—Cu—Ni as a neodymium magnet electrode (MagEle). Using HEK cells that stably express Nav1.5/Kir2.1 in which the gold-coated magnetic nanoparticles prepared in (Reference 1-2) were introduced in advance, an intracellular recording electrode was constructed by attracting intracellular gold-coated magnetic nanoparticles to the neodymium magnet electrode (MagEle), and by penetrating the cell membrane with a neodymium magnet electrode placed above the cell (FIG. 5). The intracellular recording electrode obtained was used to record the intracellular potential change of “Stable expression HEK cells of Nav1.5/Kir2.1” (FIG. 6).

### (Reference Example 1-4) Method of Penetrating Conductive Nanoparticle Adsorbed on Magnet Electrode into Cell Membrane

In this experiment, it is shown that the intracellular recording electrode can be constructed by using the magnetic force of the magnet electrode to allow the conductive nanoparticles to penetrate from the outer surface of the cell without introducing the conductive nanoparticles into the cell in advance.

Gold-coated magnetic nanoparticles (NITmagold Cit manufactured by nanoimmunotech) mixed with PEI were added to the surface of the magnet electrode coated with an insulator on the side surface, and the nanoparticles were adsorbed to the magnet electrode over about 30 minutes.

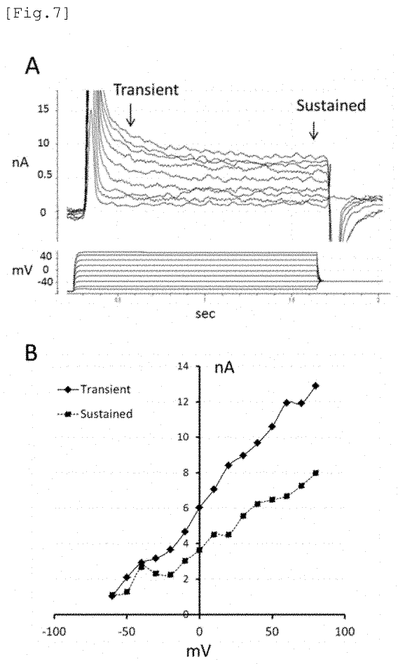

From above Nav1.5/Kir2.1 HEK cells and cultured cardiomyocyte (iCell cardiomyocyte) in a culture dish were placed on an iron plate, and the magnet electrodes were directly contacted with the adsorption surface of the conductive nanoparticles facing downward. It was observed that the magnet electrode was attracted to the iron plate below the culture dish and fixed above the cells, and the conductive nanoparticles penetrated the cell membrane by the action of PEI and remained inside the cell membrane due to the magnetic force from the magnet (FIG. 7).

Furthermore, in any cell, the intracellular potential change could be recorded by the obtained intracellular recording electrode as in the case of (1-3).

That is, an intracellular recording electrode based on a magnet electrode having adsorbed a mixture of conductive nanoparticles and PEI can be constructed without the step of previously introducing conductive nanoparticles into cells. By pressing this electrode against the cell, it was confirmed that the intracellular potential change can be recorded with very low invasiveness.

### (Reference Example 2) Method of Measuring by Applying the Principle of a Charge Amplifier

### (Reference 2-1) Preliminary Experiment for Applying the Principle of Charge Amplifier

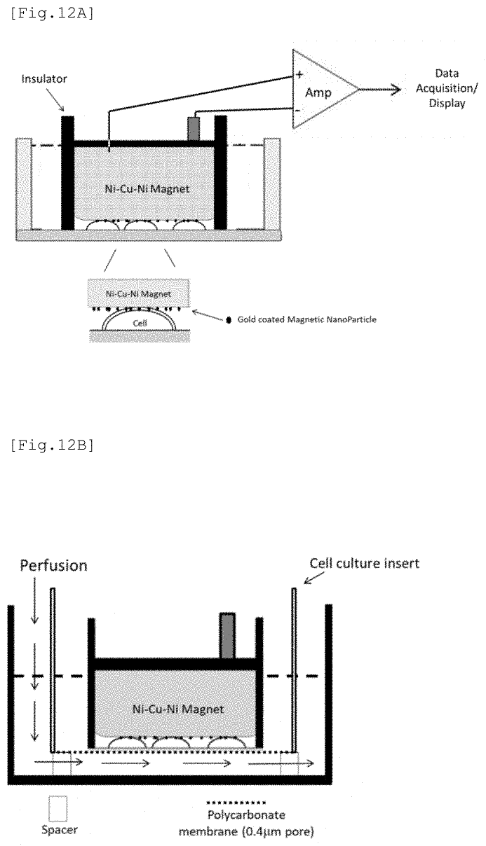

This experiment is an experiment to show that the measurement system is effective in measuring the bioelectric potential by combining the charge amplifier with nanoparticles introduced into the cell (FIG. 12).

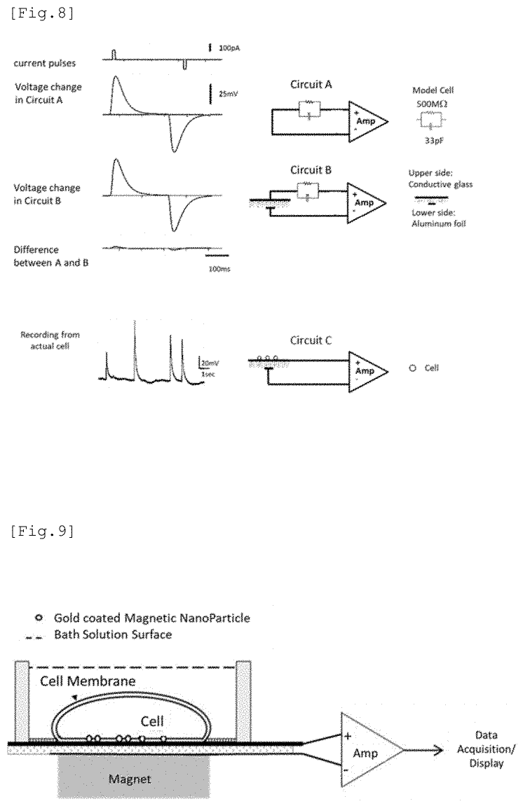

Circuit A adds a rectangular current pulse (20 ms, 120 pA) to a model cell (cell equivalent circuit: a circuit in which a 500 MΩ resistor and a 33 pF capacitor are connected in parallel) to the current-clamp mode patch-clamp amplifier Axoptch 200A used for recording. The potential change through the equivalent circuit was measured (FIG. 8 circuit A).

Following that, a conductive glass that serves as a sensor for the charge amplifier was connected between the equivalent circuit and the negative input of the amplifier. In order to make this conductive glass act as a capacitor, an aluminum foil was placed below the glass without the conductive coating (FIG. 8 circuit B).

The effect of inserting a conductive glass-aluminum foil capacitor on the voltage output was evaluated by calculating the difference between circuits A and B. Similar experiments conducted separately showed errors of 3.3% and 5.7% reduction in waveform amplitude, although other than that, it was found that the waveform itself was not affected by the filter effect.

From this, it was judged that the conductive glass-aluminum foil capacitor is effective as a sensor of the charge amplifier and can be applied to the measurement of the electrical activity (movement of charged ions inside and outside the cell) of the cells cultured on the conductive glass.

In the circuit C of FIG. 12, a circuit when an actual cell is used is shown. As a sample, the same cells (Nav1.5/Kir2.1 HEK cells) as those used in the above (Reference Example 1) were used, and the spontaneous action potential of the cells were measured using a charge amplifier to determine the intracellular membrane potential. It was confirmed that the measurement can be performed with an extracellular recording device by applying the principle of the charge amplifier (FIG. 8, circuit C).

### (Reference 2-2) Myocardial Cells Using a Charge Amplifier

In this experiment, using myocardial cells, changes in the intracellular membrane potential caused by action potentials in the cells are regarded as changes in charge through conductive nanoparticles, and this charge signal change (charged ions accompanying the generation of action potentials). It is shown that movement of Na+, K+, Ca2+, Cl− (inside and outside the cell) can be measured as a voltage signal by applying the principle of the charge amplifier (FIG. 9).

More specifically, cardiomyocyte-like cells (iCell Cardiomyocytes: CDI) were seeded on conductive glass (thickness 2 mm) and cultured using “iCell Cardiomyocytes Maintenance Medium” as a culture medium. Gold-coated magnetic nanoparticles were introduced into myocardial-like cells using Streptolysin O (SLO, Wako Pure Drug) by the following method.

That is, it was washed once with PBS (−) and replaced with nanoparticles-SLO mix (20 ul PEG-Gold coated magnetic nanoparticle, 5 μl 5×HBPS, 1 μl (1 U) activated SLO). Hold in incubator (37° C.) for 15 minutes. Subsequently, the solution was replaced with iCell Cardiomyocytes Maintenance Medium (inactivation of SLO by serum), and the experiment was carried out from the next day onward.

Similar to the circuit C of (Reference 2-1) above, the upper surface of the conductive glass covered with myocardial cells into which gold-coated magnetic nanoparticles have been introduced is connected to the input of the amplifier, and the aluminum foil placed on the lower surface of the glass is connected to the ground. A capacitor was formed from the upper surface of the conductive glass and the aluminum foil on the lower surface, and the electrical activity (movement of charged ions inside and outside the cell) of the cells cultured on the upper surface were measured (FIG. 14).

In this way, it was demonstrated that the spontaneous action potential of cells can be measured by conductive nanoparticles using the principle of the charge amplifier.

### (Reference Example 3) Charging Amplifier Mode (ChargeAmpLifier (CHAMPL) Mode) Recording of Cells with Weak Adhesive Strength

This experiment shows that cells with weak adhesion can be efficiently adhered to the surface of conductive glass, and from those weakly adhesive cells can be subjected to the intracellular action potential measurement by attracting conductive nanoparticles, pre-introduced into cells, and penetrating the cell membrane from below the conductive glass with a magnet.

### (Reference Example 3-1) Cell Seeding Method for Cells with Weak Adhesion to Conductive Glass Surface

Normal animal cells such as CHO cells efficiently adhere to the conductive glass surface, but some cells such as cardiomyocytes and HEK cells have extremely low adhesion efficiency to the conductive glass surface. When such cells are directly seeded on the surface of the conductive glass, it is extremely difficult to culture the cells to cover the entire surface of the conductive glass forming the bottom surface of the culture vessel.

Therefore, collagen is applied to the surface of the conductive glass in a grid shape in advance, and cells are seeded on the conductive glass with collagen grid shape coating and cultured until the entire surface is covered. In the place where the collagen coating film is not present, the conductive nanoparticles penetrating the cell membrane can be directly contacted with the conductive glass.

Although collagen has low conductivity, it has a high cell adhesion property and a high adhesion property to the conductive glass, so it is possible to improve the cell adhesion rate at the collagen coating film portion. Other examples of such substances include fibronectin and Poly-L-lycine, which can be used in place of collagen.

Experiments were conducted using myocardial-like cells (iCell Cardiomyocyte2, CDI) as a typical example of cells with low adhesion efficiency to the surface of conductive glass. In order to improve the adhesiveness, the cells were cultured on the surface of conductive glass coated with collagen (Collagen) in a grid pattern in advance. Gold-coated magnetic nanoparticles were introduced into myocardial-like cells and penetrated the cell membrane by a magnetic field. Sodium channels were activated by sodium channel openers such as veratridine and the resulting action potentials were recorded. This experiment was performed at room temperature.

First, gel-like collagen (Atelocollagen, Koken Co., Ltd.) is applied linearly within a circle with a diameter of about 5 mm at the center of the surface of the conductive glass. It was stretched in a grid pattern using microelectrodes with melted tips to form a grid-like collagen coating film at intervals of about 0.5 μm.

Next, cardiomyocyte-like cells (iCell Cardiomyocyte2) were seeded on the surface of the conductive glass and cultured in a cardiomyocyte-specific medium (iCell Cardiomyocytes Maintenance Medium) for about six days to form a cardiomyocyte sheet.

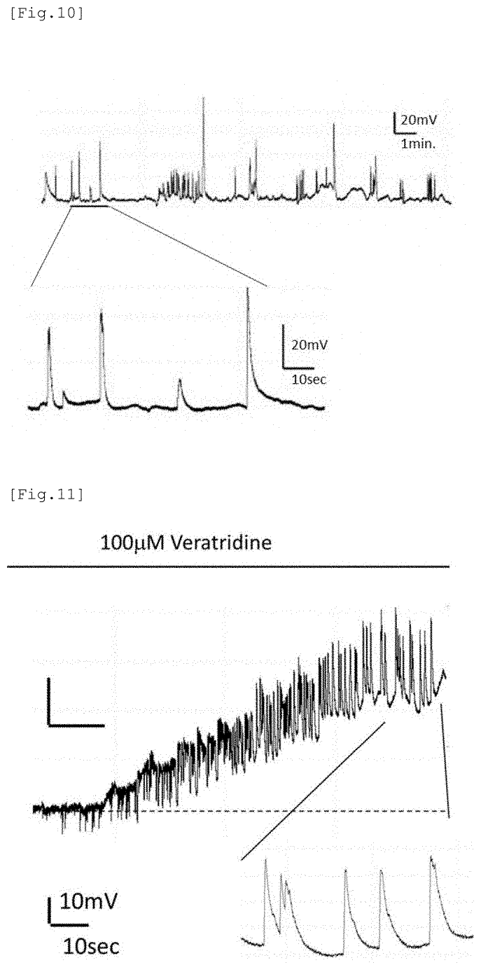

Next, using SLO, gold-coated magnetic nanoparticles were introduced into the cell. The intracellular nanoparticles were attracted by the action of a magnetic field generated by a magnet placed under the conductive glass, penetrated through the cell membrane, and brought into contact with the conductive glass to form a nanoparticles-electric glass electrode. Two days later, the culture medium was replaced with physiological saline before the experiment, and the action potentials of the cells were recorded at room temperature using the charge amplifier mode (ChargeAmpLifier (CHAMPL) mode) (FIG. 10).

However, since the spontaneous action potential could not be observed in this sample (data not shown), Veratridine (Sigma-Aldrich), which acts as a sodium channel opener, was administered to a final concentration of 100 μM in physiological saline. Action potentials evoked by veratridine were recorded. Slow depolarization was observed 40 seconds after Veratridine administration, followed by spontaneous action potentials recorded (FIG. 11).

### (Reference Example 4) Measurement of Intracellular Potential in Cultured Nerve Cells

The purpose of this example is to confirm that the intracellular potentials from cultured nerve cells in which gold-coated magnetic nanoparticles are penetrated the cell membrane according to the method of Reference Example 1, can be measured by using a magnet electrode having a capacitive potential measurement function.

### (Reference 4-1) Preparation of Cultured Nerve Cells

NG108-15 cells were used for the purpose of showing that recording is possible from cultured nerve cells. NG108-15 cells (neuroblastoma-glioma hybrid cells) were cultured in a culture medium containing DMEM (Sigma-Aldrich), HAT supplement (Thermofisher) and 10% FBS (Biowest). NG108-15 was seeded on a cover glass that was coated with 0.1% Polyethyleneimine (pH8.4, 150 mM Sodium Tetra-Borate (Wako)), and cultured in the culture medium, in which FBS concentration was reduced to 5% from the above culture medium, and the nerve differentiation was induced by adding 500 μM Ibuprofen (Wako). The following experiment was conducted five days after nerve induction.

### (Reference 4-2) Penetration of Gold-Coated Magnetic Nanoparticles into the Cell Membrane by the Magnet Electrode Method

PEI and PEG-gold coated magnetic nanoparticles were mixed and left at room temperature for three hours. This mixed solution is moved to the cell-adhesive surface of the neodymium magnet and left for additional fifteen minutes to magnetically bond the gold nanoparticles to the neodymium magnet electrode.

The above nanoparticle-bonding magnet electrode that were placed on the cultured cells were placed on an iron plate. The magnet electrode is self-sustained and fixed on the cell by the magnetic force generated between the iron plate under the cell and the magnet electrode. The gold nanoparticles on the surface of the magnet electrode were penetrated by the action of PEI so that one end was exposed inside the cell.

As a magnet electrode, a neodymium magnet with a magnetic force of 220 millitesla and a diameter of 6 mm was used. A magnet electrode-type capacitance type potential measurement device is formed in which a magnet electrode (MagEle) whose surface other than the cell contact surface is covered with parafilm is connected to a positive input, and a ferromagnetic material placed on the parafilm is connected to a negative pole. At this time, it was confirmed that the parafilm between the ferromagnetic material and the magnet electrode was made thick to ensure complete insulation (FIG. 12A). The recording was performed using the above nanoparticle-bonding magnet electrode (C-M electrode) with the added function of the capacitance-type potential measurement device.

### (Reference 4-3) Preparation of Cells Using Polycarbonate Cell Culture Insert

Here, except that the neural hybridoma cells (NG108-15 cells) were seeded and cultured in a Polycarbonate cell culture insert (pore size 0.4 μm, Thermo Scientific) instead of the cover glass used in (Reference 4-1). Conductive nanoparticles (gold nanoparticles) were prepared by the same method as in the above (3-1) and (3-2), and then the cell membrane was penetrated by the conductive nanoparticles (FIG. 12B).

The advantage of using a cell culture insert is that the cells are cultured on a polycarbonate permeable membrane with a pore size of 0.4 μm (Thermo Scientific). Even if the upper surface of the cell is covered with the magnet, the solution can be replaced through the gap below the insert. It is also applicable to observe the action of a drug and to measure the activity of ligand gated channel by administering Agonist.

### (Reference 4-4) Recording of Intracellular Potential of Cultured Nerve Cells

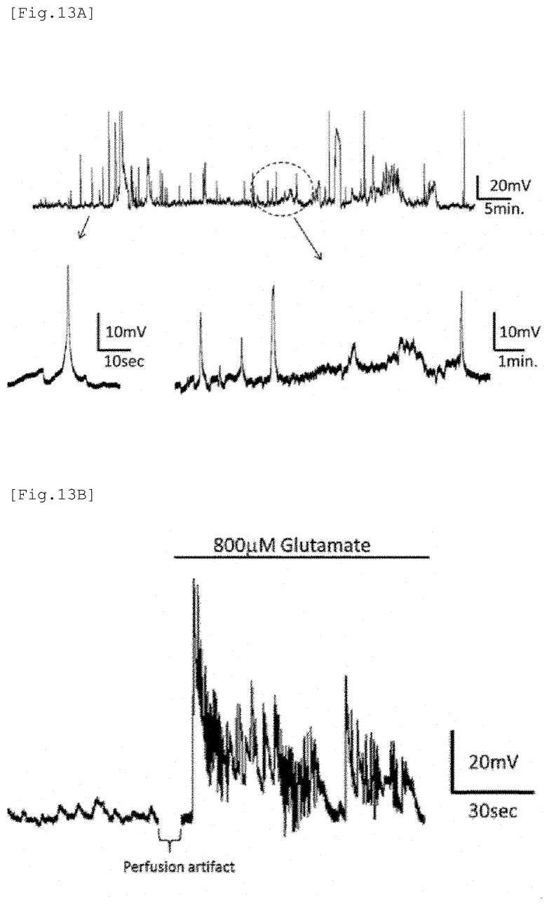

After inducing neural differentiation of NG108-15 cells for five days, the culture medium was replaced with extracellular fluid (physiological saline), and an action potential recording experiment was performed. Spontaneous action potentials were recorded by placing C-M electrode on cells seeded on coverslips. Since this method strongly depends on the spontaneous activity of cells, the degree of neural differentiation greatly affects the success of the experiment (FIG. 13A).

In addition to the spontaneous action potential, the following experiment was conducted to confirm whether the neuronal activity by the neurotransmitter glutamate can be recorded. Experiments were carried out using NG108-15 cells that had been subjected to neural differentiation induction for two days. Similar to the above experiment, C-M electrode was placed on the cells and the experiment was performed. Glutamic acid was administered to the extracellular fluid (final external fluid concentration 800 μM), and the response mediated by glutamate receptors was recorded. Glutamate activated the endogenous glutamate receptor, which was recorded as a depolarization (upward change) response of the membrane potential. The membrane potential change induced by glutamate decayed slowly due to desensitization and returned to baseline after about 90 seconds. The baseline and glutamate response artifacts on the figure were removed (FIG. 13B).

Since slow changes in membrane potential that last for several seconds can be recorded, this method can also be used to record changes in membrane potential due to ion channels activated by neurotransmitters and G channel-mediated ion channel activity.

### (Example 1) Measurement of Intracellular Potential of NG108-15 Differentiated Cells

### (1-1) Preparation of Differentiated NG108-15 Cells

The NG108-15 differentiated cells used in this experiment were prepared as follows.

Differentiated NG108-15 cells used in this experiment (Distributed by Professor Furuya, Nagoya University: FURUYA et al. (1983) Developmental Time Courses of Na and Ca Spikes in Neuroblastoma×Glioma Hybrid Cells. Developmental Brain Research, 11 229-234) does not spontaneously show an active potential in most cells, but an active potential can be induced by electrical stimulation. Furthermore, administration of the excitatory neurotransmitter glutamic acid (10 mM) can continuously induce action potentials.

The method for culturing NG108-15 cells was the same as that of Furuya et al.

More specifically, a solution containing 2% H.A.T. in DMEM (Sigma-Aldrich) containing 10% FBS (Biowest) was used as the culture medium. According to the literature, in order to differentiate NG108-15 cells into nerve-like cells, FBS in the culture medium is reduced to 2% and the intracellular cAMP concentration is increased by applying the membrane-permeable cAMP (Dibutyryl-cAMP), but in this protocol, it was replaced with 10 uM forskolin. The culture medium was changed at intervals of 2 to 3 days.

(1-2) The Intracellular Potential Measurement Experiment Using the Patch Clamp Method was Performed to Confirm that the NG108-15 Cells are Differentiated as Nerves (Electrical Stimulation and Acquisition of Responsiveness to the Excitatory Neurotransmitter Glutamic Acid).

This experiment verified that the intracellular potential was induced by glutamate in NG108-15 differentiated cells adjusted in (1-1) using the conventional patch-clamp method.

More specifically, using a glass pipette filled with an intracellular electrolyte solution containing 10 mM glutamate to differentiated NG108-15 cells, the cell membranes were tightly adhered and the glass pipette and cells were electrically integrated. It was then recorded that the added glutamate induced a slow depolarization and action potentials (FIG. 14).

(1-3) Fabrication of Integrated Electrode with Nano-Protrusion Structure at the Tip

A magnet electrode (Magele) was used as the electrode, and commercially available citric acid-stabilized magnetic gold nanoparticles (manufactured by nanoimunotech) were used as the conductive nanoparticles.

More specifically, 0.5 μl PEI (10 mg/ml), 20 ul gold-coated magnetic nanoparticles (NITmagold Cit manufactured by na noimmunotech), and 5 μl of 5 times the concentration HBPS (final concentration of calcium and magnesium is 1 mM) were mixed. It was kept at room temperature for 30 minutes or more. The recording surface of the magnet electrode is fixed upward, the mixed solution is dropped onto the recording surface, adsorbed by magnetic force, and the solution is evaporated at room temperature (Magele).

As shown in the middle drawing of FIG. 3, the entire side surface of the electrode was inserted into a silicon tube (Tyg on) of an insulator to cover it, and the area in contact with the extracellular fluid was further covered with aluminum foil as a ground material. Through the above process, the integrated electrode of the present invention was produced.

### (1-4) Intracellular Recording Using an Electrode Having a Nano-Protrusion Structure at the Tip

This experiment shows that the intracellular potential of differentiated NG108-15 cells can be measured using an electrode in which a conductive nano-protrusion structure is integrated with the tip prepared in (1-3).

The integrated electrode was pressed onto the differentiated NG108-15 cells using a manipulator (FIG. 2), and the integrated electrode was fixed in a state where the nano-protrusion at the tip penetrated the cell membrane and the cells were not crushed. The pressure of the manipulator at that time was about 280 g/cm² from the result of actually pressing it against the scale and measuring it.

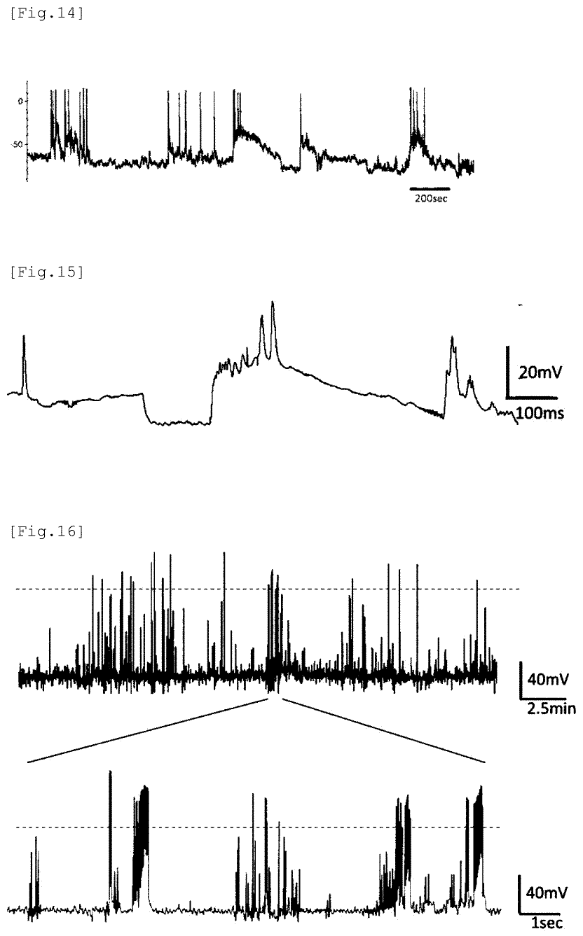

After activating the differentiated NG108-15 cells with 10 mM glutamate, the intracellular potential was measured with an integrated electrode fixed with a manipulator. Action potentials and slow depolarization were recorded, similar to the recordings obtained by the patch clamp in (1-2) above (FIG. 15).

### (Example 2) Measurement of Intracellular Potential of NG108-15 Cells Activated by Veratridine

In this experiment, differentiated NG108-15 cells were activated with veratridine, which is a sodium channel opener, and their intracellular potentials were measured.

### (2-1) Measure the Intracellular Potential by Magele

A culture dish in which differentiated NG108-15 cells were cultured was placed on an iron plate, and a magnet electrode (Magele) in which gold-coated magnetic nanoparticles were attracted to the tip using the same method as in Reference Example (1-3) was used as an intracellular recording electrode, and the intracellular potential of NG108-15 cells activated by veratridine was measured.

More specifically, this Magele was autonomously fixed on differentiated NG108-15 cells by magnetic force with an iron plate below the culture dish. Nanoparticles at the tip of Magele penetrated the cell membrane with the help of PEI and measured the intracellular potential. NG108-15 cells were activated by veratridine administered to extracellular fluid, and the action potential generated thereby was measured as an intracellular potential change (FIG. 16).

(2-2) Measurement of Intracellular Potential of NG108-15 Cells Using an Integrated Electrode with Conductive Gold Nanoparticles Deposited on the Surface

Since the presence or absence of magnetism does not affect the present invention, a magnet electrode (Magele) was used as the electrode, and commercially available citric acid-stabilized magnetic gold nanoparticles (manufactured by nanoimunotech) were used as the conductive nanoparticles. Gold nanoparticles and L (+)-ascorbic acid (10 mg/ml, Wako Pure Chemicals) is mixed 1:1 and about 20 μl is dropped onto the tip of a magnet electrode (Magele) (or conductive glass), heated at 75° C. for 15 minutes, and vaporized. A particle-like “conductive nano-protrusion structure” was formed at the tip of the magnet electrode. After vapor deposition, it was washed with pure water. An electrode was formed through the above process.

Here, the magnetic gold nanoparticles were adsorbed on the surface of the magnet electrode (Magele) and pressed from the outside of the cell against the cell surface by a manipulator.

First, an electrode having a holder was inserted into an insulator silicon tube (Tygon) to cover the entire side surface, and the area in contact with extracellular fluid was further covered with aluminum foil, which is a ground material (Integrated electrode). Next, gold-coated magnetic nanoparticles mixed with polyethyleneimine (PEI) were adsorbed on the surface opposite to the holder of the integrated electrode, and were pressed against the cell surface from above the differentiated NG108-15 cells by a manipulator to the tip. Gold nanoparticles were penetrated into the cell membrane and fixed to the cell surface.

NG108-15 cells were activated with 10 mM glutamic acid, and their intracellular recording potentials were measured. As a result, the similar data as above (FIG. 15) was obtained (data not shown).

### (Example 3) Measurement of Action Potential of NG108-15 Cells Activated by K⁺ Channel Blocker

In this experiment, an electrode (polyethyleneimine (PEI) coating) having a conductive nano-protrusion structure at the tip, which was produced by the same method as (2-2) above, was used. The action potential of NG108-15 cells activated by the K⁺ channel blocker is measured.

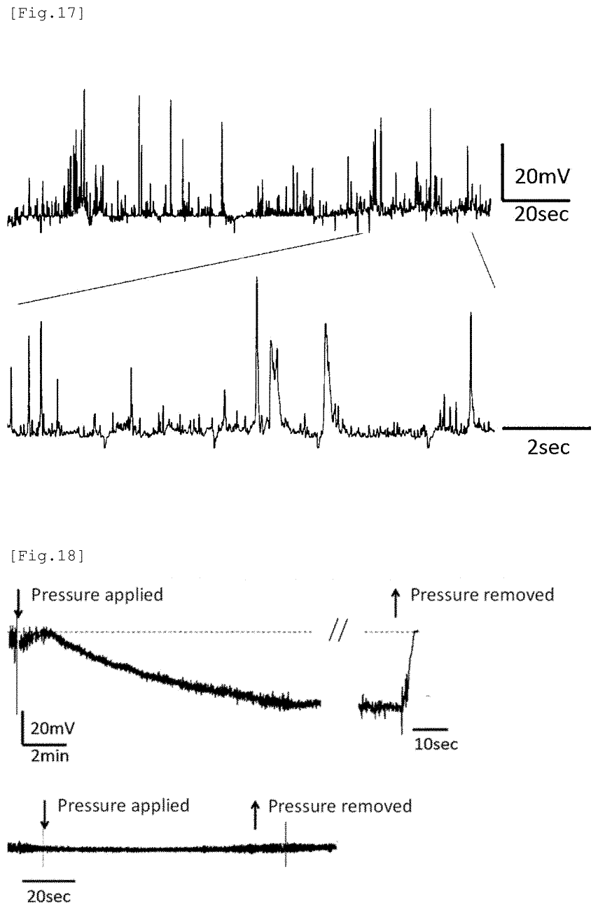

More specifically, as in (2-2) above, a magnet electrode having magnetic gold nanoparticles adsorbed on the surface is pressed against the cell surface from outside the cell by a manipulator, and the gold nanoparticles at the tip are pressed against the cell membrane. The differentiated NG108-15 cells were activated by a K+channel blocker by penetrating and fixing the magnet electrode on the cell surface, and the intracellular recording potential thereof was measured (FIG. 17).

### (Example 4) Measurement of Intracellular Potential of Undifferentiated NG108-15 Cells

In this experiment, it is shown that an electrode having a conductive nano-protrusion structure at the tip can measure the intracellular recording potential of a target cell even when both the conductor and the nano-protrusion structure at the tip are titanium.

(4-1) Fabrication of an Electrode Having a Conductive Nano-Protrusion Structure at the Tip (Titanium Electrode in which Titanium Colloid is Adhered to the Tip of a Nickel-Copper Section)

Using a method such as Suzuki (Journal of the Ceramic Society of Japan 117 (3): 381-384 (2009)) with some modifications, nanostructure irregularities were formed at the tip of the nickel-copper (6 mm diameter) section.

More specifically, it is first treated with 1% KOH for 5 minutes to create a hydrophilic surface and then washed with ddH₂O.

As solution A, 1% titanium (IV) bis (ammonium lactate) dihydroxide (TAL H, 50% Aldrich) was used. As solution B, 0.1 wt % TiO2 colloidal solution was prepared by diluting 30 wt % TiO2 colloidal (STS-01, Ishihara Sangyo Co., Ltd.) with ddH₂O to prepare a 0.1 wt % aqueous solution, and then the solution was adjusted to pH2 using HCl.

Next, solution A and solution B were alternately sprayed on the titanium surface including washing with water to grow titanium crystals, and an electrode having a titanium nanostructure at the tip portion was prepared. The spray cycle was stopped at ten cycles where nanostructured irregularities still remained on the painted surface.

### (4-2) Measurement of Resting Membrane Potential of Undifferentiated NG108-15 Cells

A titanium electrode having a titanium nanostructure at the tip portion produced by the method (4-1) is covered with an insulator and a ground by the same method as (1-3) above, and is attached to a holder. This electrode was attached to a manipulator and pressed against undifferentiated NG108-15 cells by the manipulator to penetrate the cell membrane, and the resting membrane potential was measured while pressurizing. By releasing the pressurization, the recorded membrane potential disappeared (the state before pressurization was reached) (FIG. 18, upper figure).

When the same experiment was performed in the absence of cells, there was no change in the potential (FIG. 18, lower figure), so the recorded membrane potential was judged to be derived from cells.

### (4-3) Method of Using SKILTITAN as a Nanostructure at the Tip of Titanium

Instead of adhering the Titanium colloid of (4-1) above to the tip of the titanium piece, SKILTITAN (Kin. Dai Co., Ltd.) is sprayed on the electrode surface to create a nanostructure at the tip of the titanium piece. Then, a titanium electrode similar to the above (4-1) was produced.

When the intracellular resting membrane potential of undifferentiated NG108-15 cells was measured using the titanium electrode by the same method as in (4-2) above, the same data as in (FIG. 18, upper figure) was obtained (Data not shown).

### (Example 5) Examination of Other Methods for Forming a “Conductive Nano-Protrusion Structure” at the Tip of a Conductor

Here, it is shown that polyaniline nanofibers having a nanostructure made of a conductive polymer can be formed at the tip of an electrode (for example, a titanium section).

By using the method of Chiou et al. (Nature Nanotechnology 2, no. 6 (June 2007): 354-57.) with some modifications, it is possible to form nanostructures from 50 nm to 65 nm using Polyaniline Nanofibres as the “conductive nano-projection structure” at the tip of the electrode.

More specifically, aniline (Wako Junyaku) is used to form polyaniline nano-protrusion under the following conditions. Dissolve aniline with 1 M perchloric acid (HClO4, Wako Pure Chemical) to make a 10 mM aniline solution, and make a 1.5-fold ratio ([aniline]/[APS) to APS (ammonium peroxydisulfate (Wako Pure Chemical))]=1.5) and mix with this aniline solution and stir with a stirrer at 0-5° C. for 24 hours to form Polyaniline nanofibres on a titanium plate (e.g. 0.5 mm thick, 1 mm −1 cm length). A titanium plate having the obtained Polyaniline nano-protrusions on the surface is bonded to the tip of the electrode.

### (Example 6) Construction of “Capacitive Potential Measuring Device” with Holder

By applying the “capacitive potential measuring device method” (Japanese Patent Application No. 2018-87689) developed by the present inventors, a compact “capacitive potential measuring device” with a holder (FIG. 3, right) was constructed. Here, as shown in (FIG. 3, right), the ground (reference electrode) of the “capacitive potential measuring device” with a holder is integrated with the electrode by connecting it to the negative side of the measuring instrument or the ground terminal (See the description of “Clip on ground” in 4.4-4 above). This “capacitive potential measuring device” with a holder has a conductive nano (particle) structure at the tip that penetrates the cell membrane. By detecting the change in electric potential inside the cell and charging the conductor (magnet electrode, etc.) with which the conductive nano (particle) structure is in contact. The intracellular potential change is detected as the amount of potential change.

Therefore, the number per cell of the tip conductive nano (particle) structure penetrating the cell membrane strongly affects the cell membrane and the conductive glass access resistance and determines the magnitude of the recorded potential change.

More specifically, the potential change is detected as a charge change (Q) of the conductive nano (particle) structure at the tip. Q is derived by the following equation.

Q=CV (In the formula, C is the capacitance of the capacitor and V is the amount of change in potential)

Therefore, the value of V, which is a variable, is proportional to the value of Q through the magnitude of C, which is a constant. From the above, in the capacitive potential measurement device method, the Relative Voltage Unit (RVU) is used as the relative change in the membrane potential, instead of using the unit indicating the absolute value of millivolts.

### (Example 7) Treatment Method for Ensuring Adhesion of Cells to Conductive Glass

This experiment describes the study of adhesion promotion methods when cells need to adhere to a conductive glass surface.

Generally, as a method for adhering cells to the glass surface and promoting culture, a method of coating the glass surface with an extracellular matrix such as collagen, or alternatively, in order to suppress the repulsion between the negatively charged glass surface and the cell membrane lipid, a method of poly-L-lysine treatment is required to positively charge the glass surface is used. In the nanoTouch method of the present invention, since cells need to be directly adhered to the conductive glass without coating, a special cleaning treatment is required for the surface of the conductive glass.

First, since cleaning with a strong acid or cleaning with an organic solvent is used for normal glass surface cleaning, the conductive glass (FTO, ITO) surface was similarly subjected to strong acid cleaning and organic solvent cleaning. However, strong acid washing and washing with organic solvents, such as acetone and isopropyl alcohol, did not promote the adhesion of cells to conductive glass.

After trying various solvents, finally cleaning the surface of conductive glass (FTO, ITO) with an alkaline solution was the most effective for cleaning the surface of conductive glass, and cell adhesion and culture are possible with good reproducibility.

More specifically, when normal oil stains on a surface of the conductive glass are removed with an alkaline detergent (>pH10), an alkaline solution is applied to the surface of the conductive glass in the same manner. Repeat rubbing with a brush until you can see that the glass surface does not repel water. Finally, wash with ddH2O three times or more to remove the detergent. It was confirmed that KOH, alkaline detergent, etc. are very effective as alkaline solutions.

This method is effective not only for conductive glass but also for cell culture on a glass coverslip in general. In particular, when inducing the differentiation of neuroblasts such as SH-SY5Y and when culturing iPS cardiomyocytes, coating with extracellular matrix such as collagen and fibronectin is usually required. If it is treated with an alkaline solution, it can be cultured without coating.

This method is particularly effective not only for HEK cells and CHO cells, but also for cell lines that have been found to be difficult to adhere to the glass surface. Its effectiveness was confirmed in NG108-15 cells, SH-SY5Y cells, iPS cardiomyocytes, etc. (data not shown).

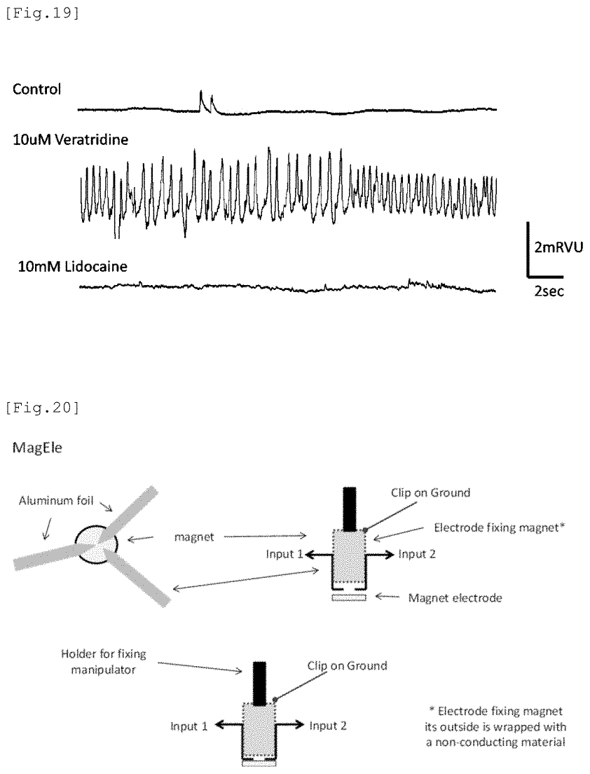

### (Example 8) Measurement of Action Potential of SH-SY5Y Cells Differentiated with Retinoic Acid (RA) (FIG. 19)

### (7-1) Differentiation and Culture of SH-SY5Y Cells by RA

In this experiment, a magnet electrode with a mixture of PEI and gold-coated magnetic nanoparticles adhered to the magnet electrode was used, the magnet electrode was fixed to a manipulator, and an electrode (Handynano electrode) produced by applying pressure from above with the manipulator was used. The intracellular potential of SH-SY5Y cells, which are human neuroblast cell lines differentiated with retinoic acid (RA; Fujifilm, 182-01111), was measured.

More specifically, SH-SY5Y cells were first seeded in a circular cover glass with a diameter of 12 mm, and the culture medium DMEM: F12=1:1 (DMEM (Sigma Aldrich, D5796), Ham's F-12 (sigma-Aldri ch, N4888)), 5% FBS (BioWest), NEAA (MEM non-essential amino acid solution (100×) (Sigma-Aldrich, M7145)) was used for culturing. From the day after seeding, 10 μM RA was allowed to act for 10 days to differentiate SH-SY5Y cells into nerve-like cells.

The surface of the cover glass was previously cleaned with an alkaline detergent as described in Example 6. Normally, culturing human neuroblast cell lines that are difficult to adhere directly to the glass surface requires surface treatment of the glass surface with an extracellular matrix such as collagen. When the alkaline solution washing method is used, human neuroblasts can be cultured because they adhere to the glass surface without surface treatment.

### (7-2) Measurement of Intracellular Potential of Differentiated SH-SY5Y Cells Activated by Veratridine

A circular cover glass, in which differentiated SH-SY5Y cells were cultured, was placed on an iron plate, and a magnet electrode with a PEI-gold-coated nanoparticles mixture magnetically attached to the tip prepared in (1-3) was pressed by a manipulator. The Handynano electrode was used as an intracellular recording electrode, the intracellular potential of differentiated SH-SY5Y cells activated by Veratridine was measured.

More specifically, using this Handynano method, the differentiated SH-SY5Y cells were fixed using a manipulator above the culture dish, and appropriate pressure (around 300 g) was applied. SH-SY5Y cells were activated by Veratridine administered to extracellular fluid, and the action potential generated thereby was measured as an intracellular potential change (FIG. 19).

As a result, it was possible to verify that SH-SY5Y differentiated cells differentiated into nerve-like cells and acquired responsiveness to Veratridine stimulation using the capacitive potentiometric method.

### (Example 8) Measurement of Intracellular Potential in Differentiated Nerve Cell Line Using nanoCharge Multi-Electrode

### (8-1) Preparation of Multi-Electrode Using MagEle

When manufacturing multiple electrodes using MagEle, two types of magnets, a fixed magnet, and an electrode magnet, are used. Wrap the fixed electrodes in a non-conductive material such as parafilm, and attach strip-shaped electrodes made of 2 to 4 pieces of aluminum foil.

This aluminum foil electrode is fixed by magnetic force to the electrode magnet from below, the side surface of the electrode magnet other than facing the cells is wrapped with a non-conductive material such as parafilm to insulate it, and “Clip on ground” is attached above the fixed magnet. A holder for fixing the manipulator is attached (FIG. 20).

(8-2) Fabrication of nanoCharge Multi-Electrode Using Conductive Glass Electrode (1)

Multi-electrode recordings were made using conductive glass electrodes instead of MagEle.

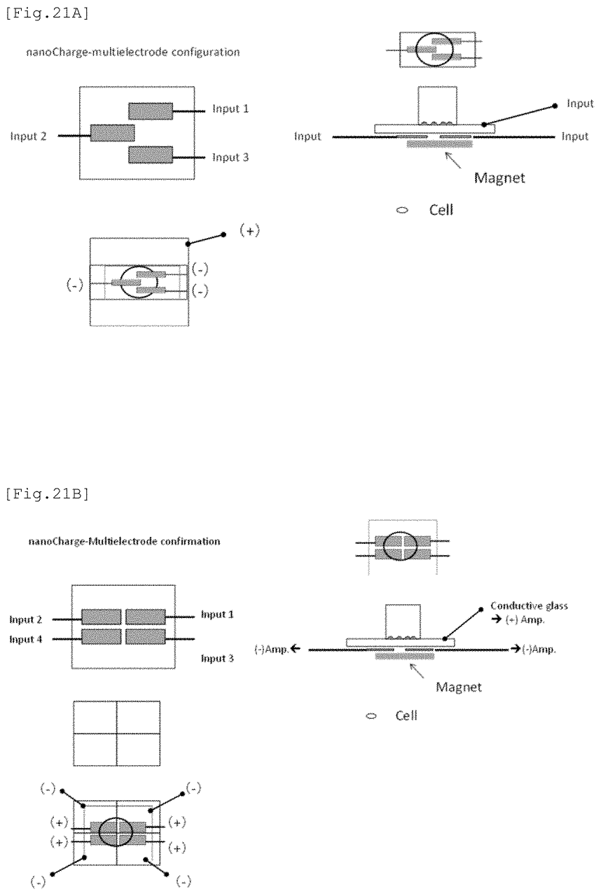

In the usual capacitive potential measurement device method, a capacitor formed between the conductive surface for culturing cells and the conductive region made of aluminum foil or the like installed on the lower surface of the conductive glass is used as a detector of the charge amplifier. Also, in the multi-electrode structure, a plurality of aluminum foil electrodes produced in the same manner were used as a plurality of capacitors, and a nanoCharge multi-electrode was manufactured using ITO conductive glass having a thickness of 0.7 to 1 mm (FIG. 21A). By fixing a plastic ring on the conductive glass (ITO) with an adhesive and filling it with a culture solution, it was possible to culture the test cells (FIGS. 21A and 21B).

Here, since the thickness of the conductive glass affects the magnitude of the recorded signal and the lateral diffusion, it is desirable that the thickness of the conductive glass is as thin as possible in order to ensure the strength of the conductive glass.

(8-3) Fabrication of nanoCharge Multi-Electrode Using Conductive Glass Electrode (2)

In the capacitive potential measurement device method, a capacitor is formed from the conductive glass on the surface and the conductive metal foil below, and the potential difference between the electrodes is measured. Therefore, when multiple electrodes are used, one conductive glass electrode and a plurality of lower electrodes are used. The electrodes and (+) (−) electrode pairs are formed, and the recording baseline tends to be unstable.

As one of the improvement methods, a plurality of conductive glasses was formed on one glass. More specifically, the conductive glass is scratched in order to block the sustainability of the conductivity. For example, by scratching the cross, four conductive glass electrodes can be formed on one piece of glass. Such a plurality of conductive glasses can form one conductive glass (FIG. 21B). As a result, the conductive glass corresponding to the lower electrode and the electrode pair are formed, so that the baseline is stable and then a stable recording signal can be obtained. In this embodiment, forming a plurality of conductive glasses on one glass is achieved by damaging the conductive glass. It can be extended to MEA (Multi Electrode Array) by forming a pattern of ITO and FTO coatings.

### (8-4) Recording of Action Potentials of NG108-15 Cells Differentiated by Forskolin Treatment

Recording was performed from NG108-15 neurons differentiated by 2% FBS, 10 μM Forskolin treatment using the 3-electrode MagEle prepared in (8-1) above (FIG. 22).

Since each of the three channels shows a different activity pattern, it can be seen that each of the three channels records signals from different cell groups. It was observed that the activity of all nerve cell groups was increased by the action of 10 uM Ach.

### (Example 9) Measurement of Intracellular Potential in iPS-Derived Cardiomyocytes Using 2-Electrode MagEle

Frozen cardiomyocytes derived from iPS were purchased from Myoridge Co., Ltd. and cultured according to the manual. Prior to the measurement of the intracellular potential, observation under a microscope confirmed that at least some cells were contracting regularly.

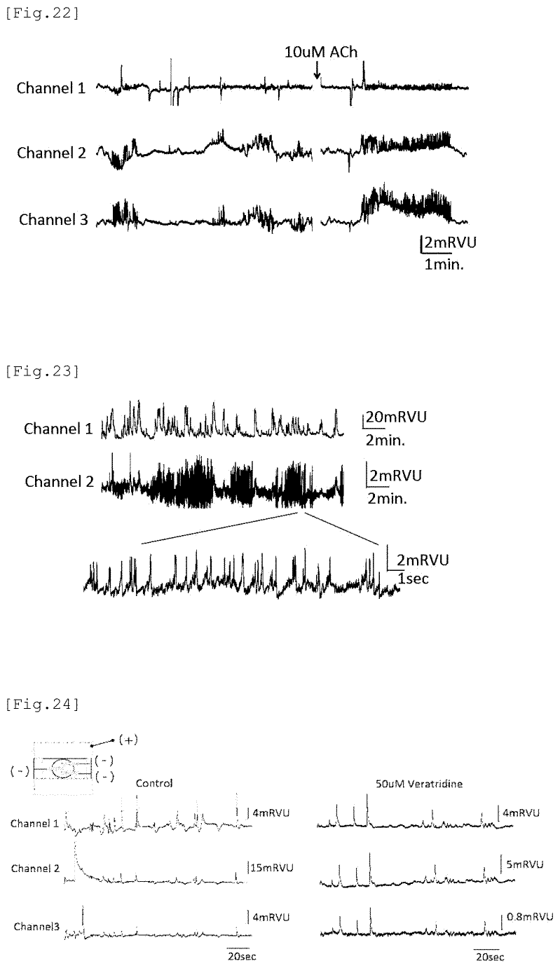

The intracellular potential of iPS-derived cardiomyocytes was recorded using the 2-electrode MagEle prepared according to (8-1) above. Significantly different responses were recorded on channels 1 and 2. Spontaneous action potentials were recorded in channel 2 at about 0.5 Hz (FIG. 23, figure below).

### (Example 10) Measurement of Intracellular Potential in a Nav1.5, Kir2.1-Expressing HEK Cell Line Using a Three-Electrode Conductive Glass Electrode

Using the 3-electrode conductive glass electrode produced in (8-2) above as a 3-electrode capacitive potential measurement device, the intracellular recording was performed on the HEK cell line expressing Nav1.5 and Kir2.1 cultured on ITO conductive glass (FIG. 24).

The Nav1.5, Kir2.1-expressing HEK cell lines used here were cultured on ITO conductive glass, and conductive nanoparticles (Gold-coated magnetic nanoparticles) were introduced into cells using PEI.

### (Example 11) Measurement of Intracellular Potential Using a MagEle Electrode Self-Supporting with a Ring-Shaped Magnet

(11-1) Measurement of Potential Change in Neuroblasts by Light Stimulation Passing Through the Center of a Ring-Shaped Magnet from Below (FIG. 26, Left)

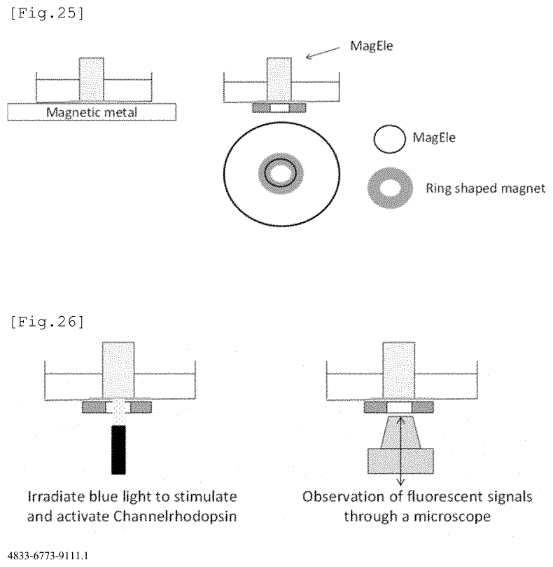

When measuring the intracellular potential by penetrating the cell membrane with a MagEle electrode that has conductive nanoparticles adsorbed on the tip placed above the cultured cells, MagEle is usually self-supported by the magnetic metal placed (Magnetic iron plate) below the cultured cells. By using a ring-shaped magnet instead of the magnetic metal, MagEle can be similarly fixed and self-supporting, and a path for directly looking at the cultured cells, to be examined below MagEle from the center of the ring-shaped magnet can be secured. Therefore, it becomes possible to project light directly onto the cells (FIG. 25).

Using the light protrusion path, it is possible to measure the intracellular potential change by applying the light stimulation.

More specifically, a ChRWD (Channel rhodopsine 1 and 2 chimera with weakened desensitization, Wang et al.) expression vector is introduced into a neuroblast cell line (NG180-15 or SH-SY5Y cells) to express ChRWD to make it sensitive to blue light stimulation, and the neuroblast cells are differentiated into nerve cell-like cells and used in experiments. LED Driver, 470 nm M470F1 (Thorlabs) is used for blue light stimulation.

Neuroblasts are cultured on a 12 mm diameter cover glass, NG180-15 cells are treated with 2% FBS and 10 uM Forskolin, or SH-SY5Y cells are treated with 10 uM Retinoic acid and cultured for 1 week to 10 days to differentiate. By placing a blue light-sensitive neuroblast cell line in a container and placing it in a container, from above, a magnet electrode having PEI-mixed conductive nanoparticles adsorbed by magnetic force is placed, and the magnet electrode is fixed upright by a ring-shaped magnet placed below the container.

Here, the ring-shaped magnet must have a sufficient inner diameter and must not interfere with the irradiation of blue light. Since the diameter of the blue LED cable used is 2 mm, the inner diameter must be at least 2 mm when inserting the LED cable into a ring-shaped magnet. At the same time, the ring-shaped magnet needs to support the above magnet electrode, so the inner diameter must not be too large. In order to secure the fixation by a sufficient magnetic force, it is necessary to secure a region where the magnet electrode and the ring-shaped magnet firmly overlap each other. That is, if the diameter of the magnet electrode is 6 mm, it is desirable that the inner diameter of the ring-shaped magnet is 4 mm or less.

A blue light stimulus is applied to a differentiated blue light stimulus-sensitive neuroblast cell line, and changes in intracellular potential are observed.

### (11-2) Simultaneous Recording of Intracellular Potential and Intracellular Calcium Dynamics (FIG. 26, Right)

In the intracellular potential recording method of cultured cells using a ring-shaped magnet and a magnet electrode, intracellular calcium measurement using a fluorescent reagent is also performed at the same time. At that time, since the magnet electrode is installed above the cell, an inverted microscope is used. Since the distance between the cell container and the lens located below is limited, it is important to keep the thickness of the ring magnet as thin as possible (2 mm or less).

Next, since 492 nm light is emitted at right angles to the magnet electrode through the objective lens, the surface of the magnet electrode is treated with a black conductive paint in order to reduce the error (Artifact) due to direct reflection. The peak SPR wavelength of gold-coated magnetic nanoparticles (NanoImmunotech) penetrating the cell membrane surface is 539±5 nm, so to avoid interference, the intracellular calcium measurement fluorescent reagent is Cal-590 AM (Ex/Em (nm) 573/587, AAT Bioquest) is used.

More specifically, 50 pg of Cal-590 is dissolved in 10 μl of DMSO, and 1 μl of Cal-590 is mixed with 100 μl of assay buffer and 1 mL of HEPES to prepare a solution. The solution is allowed to act on cultured cells, kept at 37° C. for 30 minutes to 1 hour, and the experiment is carried out at room temperature.

In addition, by applying this method, in addition to the intracellular calcium dynamics, changes in the membrane potential can be performed in parallel.

### INDUSTRIAL APPLICABILITY

The present invention is particularly useful for drug discovery screening because it can easily and accurately measure the intracellular potential. It is predicted that not only cultured cardiomyocytes but also cultured neurons will make a dramatic contribution to in vitro electrophysiological research. Furthermore, since the electrode of the present invention can act as a terminal (“in vivo prep”) that directly contacts a tissue or organ of a living body, clinical application can be also expected.

1. An intracellular recording electrode comprising a conductor having a nano-protrusion structure at the tip, wherein the nano-protrusion structure penetrates the cell membrane of a target cell; and
wherein the conductor is a magnetic electrode (Magele) having conductive nanoparticles as the nano-protrusion at the tip or is a conductor containing a non-magnetic conductive substance and having a nano-protrusion formed at the tip, and the conductor is installed at a fixed position on the upper surface of a cell and is subjected to mechanical uniform pressure from above.

2. The intracellular recording electrode according to claim 1, wherein the intracellular recording electrode forms a multi-electrode capacitive potential measuring device,
wherein the multi-electrode capacitive potential measuring device is made of capacitors formed between positive and negative electrodes: the positive electrodes are connected to the conductor; and the negative electrodes are connected to conductive plates (metal foils) placed on via an insulator.

3. The intracellular recording electrode according to claim 1, wherein the conductor is integrated with a holder that holds the conductor.

4. The intracellular recording electrode according to claim 3, wherein the holder is fixed to a manipulator capable of adjusting the degree of pressurization on the cell surface.

5. The intracellular recording electrode according to claim 1, wherein the entire side surface of the conductor body is covered with an insulator;
one or more positive poles are connected to the conductor body; and the region of the conductor in contact with the extracellular solution is covered with the insulator, wherein negative poles are connected to a region that does not come into contact with the extracellular solution to form an electric potential recording circuit.

6. The intracellular recording electrode according to claim 1, wherein the intracellular recording electrode comprises a magnetic electrode (MagEle), and the magnetic electrode has a nano-protrusion at a tip thereof, and the nano-protrusion portion at the tip penetrates a cell membrane of a target cell, wherein the magnetic electrode is set in position on a cell upper surface under mechanical uniform pressure from above or under magnetic force with a magnet or magnetic metal below a container; an entire side surface of the magnetic electrode is covered with an insulator; a plurality of positive poles are connected to the magnetic electrode; a region of a positive pole surface which makes contact with extracellular solution is further covered with an insulator material; and a negative pole is connected to an insulator surface in a region that does not make contact with extracellular solution; thus forming a multi-electrode capacitive potential measuring device with electrode pairs themselves forming capacitors.

7. The intracellular recording electrode forming a multi-electrode capacitive potential measuring device according to claim 2, wherein the multi-electrode capacitive potential measuring device is made of capacitors formed between positive electrodes and negative electrodes; and the positive electrodes are connected to the surface of conductive glass electrode having the cultured target cells into which the conductive nanoparticles have been introduced in advance; and the negative electrodes are connected to conductive electrodes composed of a plurality of metal foils that are placed on the lower surface of non-conductive side of conductive glass.

8. The intracellular recording electrode according to claim 6, wherein by breaking the continuity of the conductive glass surface, that are connected to the positive electrodes, to make sections that correspond to each positions of the multiple negative electrodes to form multiple condenser pairs.

9. The intracellular recording electrode, according to claim 7, characterized in that for promoting cell adhesion to a conductive glass surface the surface of the conductive glass is washed with an alkaline solution having a pH of 10 or more, followed by washing the conductive glass with ddH₂O at least twice to remove the alkaline solution.

10. A method for measuring the intracellular potential of target cells or its potential change, comprising the step of using the intracellular recording electrode of claim 1.

11. The method for measuring the intracellular potential of target cells or the potential change according to claim 10, comprising a step of:
using an intracellular recording electrode forming a multi-electrode capacitive potential measuring device, wherein the multi-electrode capacitive potential measuring device has capacitors formed between positive electrodes and negative electrodes; and the positive electrodes are connected to the surface of conductive glass electrodes having the cultured target cells into which the conductive nanoparticles have been introduced in advance; and the negative electrodes are connected to conductive electrodes composed of a plurality of metal foils that are placed on the lower surface of non-conductive side of conductive glass; and
treating the conductive glass surface to promote cell adhesion to the glass surface by cleaning the glass surface with an alkaline solution having a pH 10 or more, and washing with ddH₂O at least twice to remove the alkaline solution.

12. (canceled)

13. A method for measuring the intracellular potential and a change in the intracellular potential of target cells using the intracellular recording electrode according to claim 1, wherein the nano-protrusion structures at the tip of a magnet electrode (Magele) penetrate the cell membrane above the target cells, wherein light stimulation is applied on the target cells from the light projection path in the center of a ring-shaped magnet installed on the lower surface of the cover glass to which the cells, that express the photostimulatory reactive substance, are adhered.

14. A method for measuring the intracellular potential and a change in the intracellular potential of a target cells using the intracellular recording electrode according to claim 1, and measuring the intracellular calcium dynamics or the change in membrane potential of the target cells by observing the fluorescence emitted from the target cells through the fluorescence observation pathway in the center of a ring-shaped magnet installed on the lower surface of the cover glass adhered with the target cells, wherein the nano-protrusion structures at the tip of a magnet electrode (Magele) penetrate the cell membrane above the target cells, wherein the fluorescence emission is applied on the target cells from the fluorescence emission projection path in the center of the ring-shaped magnet installed on the lower surface of the cover glass to which the cells, that express the photostimulatory reactive substance, are adhered.
